(19) United States
(12) Patent Application Publication
(10) Pub. No.: US 2025/0090539 A1
(43) Pub. Date: Mar. 20, 2025

(54) COMBINATION TREATMENT OF CANCERS

(71) Applicant: Repare Therapeutics Inc., St-Laurent (CA)

(72) Inventors: Michal Zimmermann, Pointe-Claire (CA); Anne Roulston, Montreal West (CA); Julia Yang, Cambridge, MA (US); Danielle Ulanet, Cambridge, MA (US)

(21) Appl. No.: 18/888,290

(22) Filed: Sep. 18, 2024

Related U.S. Application Data
(60) Provisional application No. 63/583,510, filed on Sep. 18, 2023.

(51) Int. Cl. A61K 31/5377; A61K 31/7068; A61P 35/00

(52) U.S. Cl. CPC A61K 31/5377; A61K 31/7068; A61P 35/00

(57) ABSTRACT

Provided herein are, inter alia, methods and compositions for the treatment of cancer. The methods include administering to a subject in need thereof a therapeutically effective amount of Ataxia-telangiectasia and RAD-3-related protein (ATR) kinase inhibitors (e.g., camonsertib), pharmaceutically acceptable salts thereof, or pharmaceutical composition containing the same, and an antimetabolite (e.g., gemcitabine), pharmaceutically acceptable salts thereof, or pharmaceutical composition containing the same.

### TECHNICAL FIELD

The present application relates to combinations of Ataxia-telangiectasia and RAD-3-related protein (ATR) kinase inhibitors, pharmaceutically acceptable salts thereof, or pharmaceutical composition containing the same, and antimetabolites, pharmaceutically acceptable salts thereof, or pharmaceutical composition containing the same, and their use in the treatment of a disease or condition, such as cancer.

### BACKGROUND OF THE INVENTION

DNA is frequently damaged either by various endogenous and exogenous factors, such as ultraviolet (UV) radiation, ionizing radiation, chemical agents and if not repaired or incorrectly repaired, become lethal to the cells (De Bont and van Larebeke, Mutagenesis, 19: 169-185 (2004); Jackson and Bartek, Nature, 461: 1071-1078 (2009)). Cells respond to this DNA damage by activating a complex but distinct network of signalling pathways collectively termed the DNA Damage Response (DDR) that repair damage to maintain the integrity of the genome and prevent the development of diseases such as cancer. Mutations and dysregulation of the DDR mechanisms can lead to inefficient DNA damage resulting in genomic instability and eventually cancer development.

One major DDR protein that acts as a key cell cycle checkpoint is the ataxia telangiectasia mutated and rad3-related (ATR) kinase, related to the family of phosphoinositide 3-kinase-related protein kinases (PIKKs). ATR is activated by single stranded (ss) DNA lesions caused by stalled replication forks or during nucleotide excision repair but is also activated by double strand breaks following DNA end resection during homologous recombination. ATR is recruited to sites of DNA damage by binding to the RPA protein that coats ssDNA along with an accessory factor called ATR-interacting protein (ATRIP). The ATR/ATRIP complex is then activated by recruitment of additional factors in the 9-1-1 complex (RAD 9, RAD1, and HUS1) which subsequently recruits the TOPBP1 protein and represents critical steps for activation of the downstream phosphorylation cascade that results in cell cycle arrest. The primary target for ATR kinase is CHK1, which when phosphorylated, targets both cdc25 proteins and Wee1 resulting in inhibition of cyclin-dependent kinase activity and cell cycle arrest in S-phase or in G2/M.

The ATR kinase also acts with ATM (“ataxia telangiectasia mutated”) kinase and many other proteins to regulate a cell's response to DNA damage, in the DNA Damage Response. The DDR stimulates DNA repair, promotes survival and stalls cell cycle progression by activating cell cycle checkpoints, which provide time for repair. Without the DDR, cells are much more sensitive to DNA damage and readily die from DNA lesions induced by endogenous cellular processes such as DNA replication or exogenous DNA damaging agents commonly used in cancer therapy.

Healthy cells can rely on a host of different proteins for DNA repair including the DDR kinase ATR. In some cases, these proteins can compensate for one another by activating functionally redundant DNA repair processes. On the contrary, many cancer cells harbor defects in some of their DNA repair processes, such as ATM signaling, and therefore display a greater reliance on their remaining intact DNA repair proteins which include ATR.

In addition, many cancer cells express activated oncogenes or lack key tumor suppressors, and this can make these cancer cells prone to dysregulated phases of DNA replication which in turn cause DNA damage. ATR has been implicated as a critical component of the DDR in response to disrupted DNA replication. As a result, these cancer cells are more dependent on ATR activity for survival than healthy cells.

Many cancer treatments and chemotherapeutics have a limited treatment scope and may produce undesired side effects. There is a need for new anti-cancer therapies and, in particular, effective combinations of known inhibitors with different mechanisms of action that can synergize to increase overall efficacy and treat a broader spectrum of cancers than either inhibitor alone.

### SUMMARY OF THE INVENTION

The present application relates to the use of a combination of an ATR kinase inhibitor, or a pharmaceutically acceptable salt, and an antimetabolite, or a pharmaceutically acceptable salt, in the treatment of a disease or condition, such as cancer. The methods and compositions provided herein are, inter alia, useful for the treatment of conditions associated with DNA Damage Response, such as cancer.

In an aspect, the invention provides a method of treating a cancer in a subject. The method includes administering to the subject in need thereof a therapeutically effective amount of camonsertib, or a pharmaceutically acceptable salt, and therapeutically effective amount of gemcitabine, or a pharmaceutically acceptable salt, where the therapeutically effective amount of gemcitabine is a subtherapeutic regimen of gemcitabine.

In an aspect, the invention provides a pharmaceutical composition including camonsertib, gemcitabine and a pharmaceutically acceptable excipient, where the camonsertib and gemcitabine are present in a combined synergistic amount, wherein the combined synergistic amount is effective to treat cancer in a subject in need thereof.

In an aspect, the invention provides a method of inhibiting mammalian cell proliferation, in vitro or in vivo, including contacting a cell with a combination of camonsertib and gemcitabine, wherein the camonsertib and gemcitabine are present in a combined synergistic amount, wherein the combined synergistic amount is effective to treat cancer in a subject in need thereof.

In certain embodiments, (i) camonsertib, or a pharmaceutically acceptable salt, and (ii) gemcitabine, or a pharmaceutically acceptable salt, are administered in combination either simultaneously (e.g., as a mixture), separately but simultaneously (e.g., via separate intravenous lines, or via multiple oral formulations, or a mixture of administration modes) or sequentially (e.g., one agent is administered first followed by administration of the second agent). In certain embodiments, (i) camonsertib, or a pharmaceutically acceptable salt, and (ii) gemcitabine, or a pharmaceutically acceptable salt, are administered simultaneously or sequentially. In certain embodiments, (i) camonsertib, or a pharmaceutically acceptable salt, and (ii) gemcitabine, or a pharmaceutically acceptable salt, are administered within 7 days of each other, or are administered within 24 hours of each other, or are administered simultaneously.

In some embodiments, the method includes administering between about 10 to 200 mg of camonsertib, or a pharmaceutically acceptable salt, to the subject at least once daily for three days, followed by four days without administration of camonsertib, or a pharmaceutically acceptable salt, thereof.

In some embodiments, the method includes administering in a treatment cycle between about 10 to 200 mg of camonsertib, or a pharmaceutically acceptable salt, to the subject at least once daily on days 1, 2, 15, and 16.

In some embodiments, the method includes administering in a treatment cycle between about 10 to 200 mg of camonsertib, or a pharmaceutically acceptable salt, to the subject at least once daily on days 1, 2, 3, 15, 16, and 17.

In some embodiments, the method includes administering between about 10 mg/m² to 400 mg/m² of gemcitabine, or a pharmaceutically acceptable salt, to the subject at least once a week.

In some embodiments, the method includes administering in a treatment cycle between about 10 mg/m² to 400 mg/m² of gemcitabine, or a pharmaceutically acceptable salt, to the subject at least once daily on days 1 and 15.

In some embodiments, the method includes administering in a treatment cycle between about 10 mg/m² to 400 mg/m² of gemcitabine, or a pharmaceutically acceptable salt, to the subject at least once daily on days 1 and 14.

For example, on dosing days the method can include (1) administering to the subject between about 10 to 200 mg of camonsertib, or a pharmaceutically acceptable salt, or can include (2) administering to the subject between about 25 to 175 mg of camonsertib, or a pharmaceutically acceptable salt, or can include (3) administering to the subject between about 50 to 150 mg of camonsertib, or a pharmaceutically acceptable salt thereof, or can include (4) administering to the subject between about 75 to 125 mg of camonsertib, or a pharmaceutically acceptable salt thereof, or can include (5) administering to the subject between about 80 to 120 mg of camonsertib, or a pharmaceutically acceptable salt thereof, or can include (6) administering to the subject about 10 mg per day, about 25 mg per day, about 50 mg per day, about 75 mg per day, or about 80 mg per day, about 100 mg per day, about 120 mg per day, about 125 mg per day, about 150 mg per day, about 175 mg per day, about 200 mg per day of camonsertib, or a pharmaceutically acceptable salt thereof.

For example, on dosing days the method can include (1) administering to the subject between about 10 mg/m² to 400 mg/m² of gemcitabine, or a pharmaceutically acceptable salt, or can include (2) administering to the subject between about 25 to 375 mg/m² of gemcitabine, or a pharmaceutically acceptable salt, or can include (3) administering to the subject between about 50 to 350 mg/m² of gemcitabine, or a pharmaceutically acceptable salt thereof, or can include (4) administering to the subject between about 75 to 325 mg/m² of gemcitabine, or a pharmaceutically acceptable salt thereof, or can include (5) administering to the subject between about 100 to 300 mg/m² of gemcitabine, or a pharmaceutically acceptable salt thereof, or can include (6) administering to the subject between about 125 to 275 mg/m² of gemcitabine, or a pharmaceutically acceptable salt, or can include (7) administering to the subject between about 150 to 250 mg/m² of gemcitabine, or a pharmaceutically acceptable salt thereof, or can include (8) administering to the subject between about 175 to 225 mg/m² of gemcitabine, or a pharmaceutically acceptable salt thereof, or can include (9) administering to the subject between about 200 to 225 mg/m² of gemcitabine, or a pharmaceutically acceptable salt thereof, or can include (10) administering to the subject about 10 mg/m² per day, about 25 mg/m² per day, about 50 mg/m² per day, about 75 mg/m² per day, about 100 mg/m² per day, about 125 mg/m² per day, about 150 mg/m² per day, about 175 mg/m² per day, about 200 mg/m² per day, about 250 mg/m² per day, about 275 mg/m² per day, about 300 mg/m² per day, about 325 mg/m² per day, about 350 mg/m² per day, about 375 mg/m² per day, or about 400 mg/m² per day of gemcitabine, or a pharmaceutically acceptable salt thereof.

In some embodiments, the method includes administering (i) between about 10 to 200 mg of camonsertib, or a pharmaceutically acceptable salt, to the subject at least once daily for three days, followed by four days without administration of camonsertib, or a pharmaceutically acceptable salt, and (ii) between about 10 mg/m² to 400 mg/m² of gemcitabine, or a pharmaceutically acceptable salt, to the subject at least once a week.

In some embodiments, the method includes administering in a treatment cycle (i) between about 10 to 200 mg of camonsertib, or a pharmaceutically acceptable salt, to the subject at least once daily on days 1, 2, 15, and 16 and (ii) between about 10 mg/m² to 400 mg/m² of gemcitabine, or a pharmaceutically acceptable salt, to the subject at least once daily on days 1 and 15.

In some embodiments, the method includes administering in a treatment cycle (i) between about 10 to 200 mg of camonsertib, or a pharmaceutically acceptable salt, to the subject at least once daily on days 1, 2, 3, 15, 16, and 17 and (ii) between about 10 mg/m² to 400 mg/m² of gemcitabine, or a pharmaceutically acceptable salt, to the subject at least once daily on days 1 and 14.

In certain embodiments, the cycle is performed once, twice, or three times. In some embodiments, the method includes at least 21 days, 28 days, 2 months, 3 months, 4 months, or 6 months of treatment.

In some embodiments of any of the above methods, the camonsertib, or a pharmaceutically acceptable salt thereof, is administered orally.

In some embodiments of any of the above methods, the gemcitabine, or a pharmaceutically acceptable salt thereof, is administered intravenously.

In some embodiments of any of the aspects disclosed herein, the cancer is a solid tumor. In some embodiments, the cancer is carcinoma, sarcoma, adenocarcinoma, leukemia, melanoma, renal cell carcinoma, mature B-cell neoplasms, endometrial cancer, ovarian cancer, fallopian tube cancer, primary peritoneal cancer, colorectal cancer, skin cancer, small bowel cancer, non-small cell lung cancer, melanoma, bladder cancer, pancreatic cancer, head and neck cancer, mesothelioma, glioma, prostate cancer, breast cancer, or esophagogastric cancer. In some embodiments, the cancer is ovarian, pancreatic, prostate, or lung cancer. In some embodiments, the cancer is solid tumor. In some embodiments, the cancer is a solid tumor with deleterious DNA damage response alterations.

In some embodiments of any of the aspects disclosed herein, the cancer is associated with a loss of function of ATM. In some embodiments of any of the aspects disclosed herein, the cancer is associated with ATRIP. In some embodiments of any of the aspects disclosed herein, the cancer is associated with BRCA1. In some embodiments of any of the aspects disclosed herein, the cancer is associated with BRCA2. In some embodiments of any of the aspects disclosed herein, the cancer is associated with CDK12. In some embodiments of any of the aspects disclosed herein, the cancer is associated with CHTF8. In some embodiments of any of the aspects disclosed herein, the cancer is associated with FZR1. In some embodiments of any of the aspects disclosed herein, the cancer is associated with MRE11. In some embodiments of any of the aspects disclosed herein, the cancer is associated with NBN. In some embodiments of any of the aspects disclosed herein, the cancer is associated with PALB2. In some embodiments of any of the aspects disclosed herein, the cancer is associated with RAD17. In some embodiments of any of the aspects disclosed herein, the cancer is associated with RAD50. In some embodiments of any of the aspects disclosed herein, the cancer is associated with RAD51B/C/D. In some embodiments of any of the aspects disclosed herein, the cancer is associated with REV3L. In some embodiments of any of the aspects disclosed herein, the cancer is associated with RNASEH2A. In some embodiments of any of the aspects disclosed herein, the cancer is associated with RNASEH2B. In some embodiments of any of the aspects disclosed herein, the cancer is associated with SETD2.

Other features and advantages of the invention will be apparent from the following detailed description and FIGS., and from the claims.

### BRIEF DESCRIPTION OF THE DRAWINGS

FIGS. 1A-1C show the in vitro effect of a combination of gemcitabine with camonsertib in RPE1 TP53 cells. FIGS. 1A-1B are charts showing the ZIP synergy score matrix in RPE1 TP53 wild-type and KO cells, respectively, treated with a combination of gemcitabine and camonsertib. Scores greater than 10 indicate synergy, while scores of less than −10 indicate antagonism. Dashed lines indicate doses exerting maximum synergy. FIG. 1C is a graph of the percent viability of the PE1 TP53 wild-type and KO cells treated with a combination of 0.8 nM gemcitabine and 12 nM camonsertib.

FIGS. 2A-2B are charts showing the ZIP synergy score matrix in a BRCA1mut model treated with gemcitabine at low-nanomolar concentrations (15 nM) synergizing with camonsertib 7.5 nM (2 days on (FIG. 2A) or 3 days on (FIG. 2B)). ZIP synergy scores greater than 10 indicate drug synergy and scores less than −10 indicates antagonism. Mean CTG cell viability data from 3 independent experiments were processed with SynergyFinder (https://synergyfinder.fimm.fi/; PMIDs: 35580060, 26949479). Dotted lines indicated doses exerting maximum synergy.

FIGS. 3A-3B show the synergy of gemcitabine with camonsertib in a BRCA1 mut model. FIG. 3A shows the experimental design where SUM149PT cells were either left untreated, treated with gemcitabine for 4 h, camonsertib for 1, 2 or 3 days, or the combination of gemcitabine and camonsertib. FIG. 3B is a graph of the viability of the SUM149PT treated with the indicated compounds and schedules. Data (circles) from three independent experiments is provided with mean (bars)±SD. The P value calculated with an unpaired two-tailed Student's t-test. Cell viability was read out at day 8 with a CellTiter Glo (CTG) assay.

FIGS. 4A-4B are plots showing the in vivo anti-tumor efficacy of camonsertib in combination with gemcitabine in the SUM149PT BRCA1 mutated xenograft model. The relative tumor xenograft volume (FIG. 4A) and change in body weight in mice treated (FIG. 4B) is shown for mice treated with gemcitabine at 20 mg/kg QW on Day 1 and/or camonsertib at 10 mg/kg PO on different schedules weekly for 28 days. Results are expressed as mean±SEM, N=7/group in female NOD-SCID mice. Statistical differences were established by Student's T-test (GraphPad Prism v9) with Welch's correction; ns=not significant, *p<0.05, **p<0.01.

FIGS. 5A-5E show the efficacy of camonsertib and gemcitabine in Granta-519 tumor-bearing mice. The relative tumor xenograft volume (FIGS. 5A and 5B) and change in body weight (FIG. 5C) is shown for mice treated with gemcitabine at 50 or 100 mg/kg QW on Day 1 and/or camonsertib at 5, 10 or 30 mg/kg PO 3 days on/4 days off weekly for 28 days. Results are expressed as mean±SEM, N=8/group in female NOD-SCID mice. FIGS. 5D and 5E are graphs of the red blood cell count and neutrophils count, respectively, as measured on day 25.

FIGS. 6A-6D show the efficacy and tolerability of camonsertib and low dose gemcitabine combination in a Granta-519 ATMmut xenograft model. The relative tumor xenograft volume (FIG. 6A) and change in body weight (FIG. 6B) is shown for mice treated with gemcitabine at 5 or 10 mg/kg QW on Day 1 and/or camonsertib at 10 mg/kg QD for 15 days. Results are expressed as mean±SEM, N=8/group in female NOD-SCID mice. FIGS. 6C and 6D are graphs of the red blood cell count and neutrophils count, respectively, as measured on day 15.

FIGS. 7A-7C show the efficacy and tolerability of the combination of camonsertib and gemcitabine in a Capan-1 (BRCA2 mut) pancreatic cancer xenograft model. The relative tumor xenograft volume (FIGS. 7A and 7B) and change in body weight (FIG. 7C) is shown for mice treated with gemcitabine at 50 or 100 mg/kg QW on Day 1 and/or camonsertib at 5, 10 or 30 mg/kg dosed 3 days on/4 off weekly for 20 days. Results are expressed as mean±SEM, N=8/group in NCG mice.

FIGS. 8A-8G show the efficacy and tolerability of the combination of camonsertib and gemcitabine in human patients. FIG. 8A is a flowchart of the Arm 1 and Arm 2 dosing parameters. FIG. 8B is a plot of camonsertib PK values, both alone and in combination with gemcitabine administration. FIG. 8C shows plots of neutrophils concentration when patients are treated with an intermittent dosing schedule. FIG. 8D is a graph of the duration of treatment for patients with gynecologic cancers. FIG. 8E is a graph of the ctDNA molecular response in evaluable Arm 1 and 2 patients. On-treatment time-points were analyzed between 4-12 weeks for best molecular response, and the median timepoint for data shown is 5 weeks. A similar rate of molecular response was found in both Arm 1 and Arm 2 patients. FIGS. 8F and 8G are plots of the change in size of the target lesson and change in CA-125 concentration, respectively, in a 59-year-old patient with high-grade serous ovarian cancer (HGSOC) and ATM mutation (subclonal) over the course of a 72 week treatment.

### DETAILED DESCRIPTION OF THE INVENTION

### General

The present application provides combinations of Ataxia-telangiectasia and RAD-3-related protein (ATR) kinase inhibitors (e.g., camonsertib), pharmaceutically acceptable salts thereof, or pharmaceutical composition containing the same, and antimetabolites (e.g., gemcitabine), pharmaceutically acceptable salts thereof, or pharmaceutical composition containing the same, and their use in the treatment of a disease or condition, such as cancer.

### Definitions

### General Definitions

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

### Treatment-Related Definitions

The term “aberrant,” as used herein, refers to different from normal. When used to describe enzymatic activity, aberrant refers to activity that is greater or less than a normal control or the average of normal non-diseased control samples. Aberrant activity may refer to an amount of activity that results in a disease, where returning the aberrant activity to a normal or non-disease-associated amount (e.g. by administering a compound or using a method as described herein), results in reduction of the disease or one or more disease symptoms. The aberrant activity can be measured by measuring the modification of a substrate of the enzyme in question; a difference of greater or equal to a 2-fold change in activity could be considered as aberrant. Aberrant activity could also refer to an increased dependence on a particular signaling pathway as a result of a deficiency in a separate complementary pathway.

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal. The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “ALT+ cancer,” as used herein, refers to cancers utilizing homologous recombination-based pathway called alternative lengthening of telomeres (ALT) to extend and maintain telomeres. ALT+ cells may be identified using techniques known in the art. For example, ALT+ cells exhibit one or more of ALT-associated PML bodies, heterogeneous telomere length, abundant extrachromosomal telomere repeat (ECTR), and high levels of telomere sister chromatid exchange (T-SCE). See Bryan et al., EMBO J., 14:4240-4248, 1995; Dunham et al., Nat Genet., 26:447-450, 2000; Muntoni et al., Hum. Mol. Genet., 18:1017-1027, 2009; Yeager et al., Cancer Res., 59:4175-4179, 1999; and Cesare et al., Mol. Cell. Biol., 247:765-772, 2004. ALT+ cancer (e.g., ALT+ cancer cell) may be an ALT+ mesenchymal cancer (e.g., an ALT+ mesenchymal cancer cell). Non-limiting examples of ALT+ cancers include leiomyosarcoma, liposarcoma, glioblastoma, and neuroendocrine pancreatic cancer.

The term “ATM,” as used herein, represents ATM serine/threonine kinase.

The term “ATR inhibitor,” as used herein, represents a compound that upon contacting the enzyme ATR kinase, whether in vitro, in cell culture, or in an animal, reduces the activity of ATR kinase, such that the measured ATR kinase IC₅₀ is 10 μM or less (e.g., 5 μM or less or 1 μM or less). For certain ATR inhibitors, the ATR kinase IC₅₀ may be 100 nM or less (e.g., 10 nM or less, or 1 nM or less) and could be as low as 100 μM or 10 μM. Preferably, the ATR kinase IC₅₀ is 0.1 nM to 1 μM (e.g., 0.1 nM to 750 nM, 0.1 nM to 500 nM, or 0.1 nM to 250 nM).

The term “ATR kinase,” as used herein, refers to Ataxia-telangiectasia and RAD-3-related protein kinase.

The term “ATRIP” as used herein, represents ATM and Rad3-Related-interacting protein.

The term “BRCA1,” as used herein, represents a breast cancer type 1 susceptibility gene or protein.

The term “BRCA2,” as used herein, represents a breast cancer type 2 susceptibility gene or protein.

The term “cancer,” as used herein, refers to all types of cancer, neoplasm or malignant tumors found in mammals (e.g. humans), including leukemia, carcinomas and sarcomas. Non-limiting examples of cancers that may be treated with a compound or method provided herein include prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, ampullary cancer, colorectal cancer, and pancreatic cancer. Additional non-limiting examples may include, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, and prostate cancer.

The term “carcinoma,” as used herein, refers to a malignant new growth made up of epithelial cells tending to infiltrate the surrounding tissues and give rise to metastases. Non-limiting examples of carcinomas that may be treated with a compound or method provided herein include, e.g., medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “CDK12” as used herein, represents the cyclin-dependent kinase 12 gene or protein.

The term “CHTF8” as used herein, represents the chromosome transmission fidelity factor 8 gene or protein.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

“Disease” or “condition” refer to a state of being or health status of a patient or subject capable of being treated with the compounds or methods provided herein.

The term “FZR1” as used herein, represents the fizzy and cell division cycle 20 related 1 gene or protein.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, e.g., acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “lymphoma,” as used herein, refers to a cancer arising from cells of immune origin. Non-limiting examples of T and B cell lymphomas include non-Hodgkin lymphoma and Hodgkin disease, diffuse large B-cell lymphoma, follicular lymphoma, mucosa-associated lymphatic tissue (MALT) lymphoma, small cell lymphocytic lymphoma-chronic lymphocytic leukemia, Mantle cell lymphoma, mediastinal (thymic) large B-cell lymphoma, lymphoplasmacytic lymphoma-Waldenstrom macroglobulinemia, peripheral T-cell lymphoma (PTCL), angioimmunoblastic T-cell lymphoma (AITL)/follicular T-cell lymphoma (FTCL), anaplastic large cell lymphoma (ALCL), enteropathy-associated T-cell lymphoma (EATL), adult T-cell leukaemia/lymphoma (ATLL), or extranodal NK/T-cell lymphoma, nasal type.

The term “melanoma,” as used herein, is taken to mean a tumor arising from the melanocytic system of the skin and other organs. Melanomas that may be treated with a compound or method provided herein include, e.g., acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

The term “MRE11” as used herein, represents the meiotic recombination 11 gene or protein.

The term “NBN” as used herein, represents the nibrin gene or protein.

The term “PALB2” as used herein, represents the partner and localizer of BRCA2 gene or protein.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “progression-free survival” as used herein, refers to the length of time during and after medication or treatment during which the disease being treated (e.g., cancer) does not get worse. The combination therapies of the invention can increase the likelihood of progression-free survival in a subject.

As used herein, the term “proliferation” as used in this application involves reproduction or multiplication of similar forms (cells) due to constituting (cellular) elements. The combination therapies of the invention can decrease proliferation of cancer cells.

The term “RAD17” as used herein, represents the RAD17 Checkpoint Clamp Loader Component gene or protein.

The term “RAD50” as used herein, represents the RAD50 Double Strand Break Repair Protein gene or protein.

The term “REV3L” as used herein, represents the DNA Directed Polymerase Zeta Catalytic Subunit gene or protein.

The term “RNAse H2A,” as used herein, refers to Ribonuclease H2, subunit A.

The term “RNAse H2B,” as used herein, refers to Ribonuclease H2, subunit B.

The term “sarcoma” generally refers to a tumor which is made up of a substance like the embryonic connective tissue and is generally composed of closely packed cells embedded in a fibrillar or homogeneous substance. Non-limiting examples of sarcomas that may be treated with a compound or method provided herein include, e.g., a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “SETD2,” as used herein, represents the SET domain containing 2, histone lysine methyltransferase gene or protein.

The term “therapeutically effective amount,” as used herein, means the amount of a compound or a pharmaceutically acceptable salt thereof that, in a combination of an ATR inhibitor and PARP inhibitor, is sufficient to treat cancer. Typically, a therapeutically effective amount is a subtherapeutic regimen.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

The term “subtherapeutic regimen,” as used herein, refers to a dosing regimen that is at least 5% less (e.g., at least 10%, 20%, 50%, 80%, 90%, or even 95%) than the lowest standard recommended dosing regimen of a particular compound formulated for a given route of administration for treatment of cancer. A subtherapeutic regimen of a compound may be therapeutically ineffective for the compound in a monotherapy regimen. In the methods of the invention, a therapeutically effective amount of gemcitabine is preferably a subtherapeutic regimen (e.g., a regimen that is therapeutically ineffective for gemcitabine in a monotherapy regimen). A subtherapeutic regiment may include a “subtherapeutic starting regimen” and a “subtherapeutic maintenance regiment.” A “subtherapeutic starting regiment” of a compound (e.g., gemcitabine) is lower than the lowest standard starting dosage of the same compound (e.g., gemcitabine). Similarly, a “subtherapeutic maintenance regimen” of a compound (e.g., gemcitabine) is lower than the lowest standard maintenance regimen of the same compound (e.g., gemcitabine). Typically, the subtherapeutic regimen is at least 1% of the lowest standard therapeutic regimen. In some embodiments, the subtherapeutic regimen is at most 40% of the standard recommended dose of the monotherapy. In some embodiments, the subtherapeutic regimen is at most 40% of the standard recommended dose of the monotherapy.

“Treatment” and “treating,” as used herein, refer to the medical management of a subject with the intent to improve, ameliorate, stabilize, or cure a disease or condition. This term includes active treatment (treatment directed to improve the disease or condition); causal treatment (treatment directed to the cause of the associated disease or condition); palliative treatment (treatment designed for the relief of symptoms of the disease or condition); and supportive treatment (treatment employed to supplement another therapy). Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. A disease or condition may be a cancer. In the context of treating cancer, treatment may include slowing the spread of metastasis and/or extending progression-free survival in a population of treated subjects as compared to a population of untreated subjects. Compounds of the disclosure may also be used to “prophylactically treat” or “prevent” a disorder, for example, in a subject at increased risk of developing the disorder. Non-limiting examples of cancers include, e.g., renal cell carcinoma, mature B-cell neoplasms, endometrial cancer, ovarian cancer, colorectal cancer, skin cancer (non-melanoma), small bowel cancer, non-small cell lung cancer, melanoma, bladder cancer, pancreatic cancer, head and neck cancer, mesothelioma, glioma, prostate cancer, breast cancer, and esophagogastric cancer.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the description and from the claims.

### Methods

The methods provided herein are, inter alia, useful for the treatment of cancer. In embodiments, the methods and compositions as described herein provide effective treatment for cancers associated with the DDR pathway.

The methods herein relate to the use of a combination of ATR kinase inhibitors and antimetabolites, in the treatment of a disease or condition, such as cancer.

### ATR Kinase Inhibitors

Ataxia telangiectasia mutated and Rad3-related (ATR) pathway is a critical DNA damage response, or DDR, protein that acts as both the master regulator of the response to DNA replication stress, as well as a central effector of DNA damage checkpoints. Inhibition of ATR is synthetically lethal (SL) with genomic alterations affecting DNA damage response (DDR), and could address unmet needs across major types of cancer (Weber and Ryan, Pharmacol. Therapeut., 149:124-138 (2015); Reaper et al., Nature chemicalbiology, 7:428-430 (2011); Saldivar, J et al, Nature 18:622-636 (2017); Toledo Li et al. Nature structural & molecular biology, 18:721-727 (2011)).

### Camonsertib[0003]

Camonsertib (also referred to herein as RP-3500) is a potent and selective small molecule inhibitor of ATR and has demonstrated differentiated clinical pharmacokinetics and anti-tumor activity in patients with a variety of solid tumors characterized by deficiencies in DDR (e.g., ATM loss, BRCA1/2 mutations and other novel biomarkers) (Roulston et al., Mol. Cancer Ther. 21: 245-256 (2022). As used herein, camonsertib includes the compound:

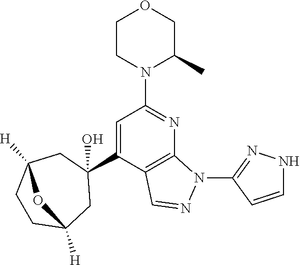 [SMILES: C[C@@H]1COCCN1c1cc([C@]2(O)C[C@H]3CC[C@@H](C2)O3)c2cnn(-c3cc[nH]n3)c2n1]

(1R,5S)-3-[6-[(3R)-3-methylmorpholin-4-yl]-1-(1H-pyrazol-5-yl)pyrazolo[3,4-b]pyridin-4-yl]-8-oxabicyclo[3.2.1]octan-3-ol, as well as all pharmaceutically acceptable salts thereof.

### Antimetabolites

“Antimetabolite” refers to an antineoplastic drug that inhibits the utilization of a metabolite or a prodrug thereof having a nucleoside derived structure.

Exemplary antimetabolites include, but are not limited to, gemcitabine, cytarabine, doxifluridine, azacitidine, decitabine, nelarabine, 2′-methylidene-2′-deoxycytidine (DMDC), tezacitabine, zalcitabine, lamivudine, 5′-deoxy-5-fluorocytidine (5′-DFCR), troxacitabine, 3′-ethynylcytidine, 2′-cyano-2′-deoxy-1-β-D-arabinofuranocylcytosine (CNDAC) or the like.

### Gemcitabine[0004]

Gemcitabine (2-Deoxy-2,2-difluorocytidine hydrochloride) exhibits cell phase specificity, primarily killing cells undergoing DNA synthesis (S-phase) and also blocking the progression of cells through the G1/S-phase boundary. Gemcitabine is metabolized intracellularly by nucleoside kinases to the active diphosphate (dFdCDP) and triphosphate (dFdCTP) nucleosides. Gemcitabine induces replication stress and dependence on ATR through two mechanisms: inhibition of DNA repair by incorporating gemcitabine nucleotides into the DNA, and inhibition of ribonucleotide reductase leading to depletion of the deoxyribonucleotide pool required for replication and repair (Fordham et al., Blood Adv., 2:1157-69 (2018); Karnitz L M et al., Mol Pharmacol. 68:1636-44 (2005)). First, gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in the concentrations of deoxynucleotides, including dCTP. Second, gemcitabine triphosphate competes with dCTP for incorporation into DNA. The reduction in the intracellular concentration of dCTP (by the action of the diphosphate) enhances the incorporation of gemcitabine triphosphate into DNA (self-potentiation). After the gemcitabine nucleotide is incorporated into DNA, only one additional nucleotide is added to the growing DNA strands. After this addition, there is inhibition of further DNA synthesis. DNA polymerase epsilon is unable to remove the gemcitabine nucleotide and repair the growing DNA strands (masked chain termination). In CEM T lymphoblastoid cells, gemcitabine induces internucleosomal DNA fragmentation, one of the characteristics of programmed cell death.

The term “gemcitabine” as used herein includes the compound:

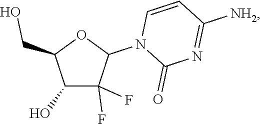 [SMILES: Nc1ccn(C2O[C@H](CO)[C@@H](O)C2(F)F)c(=O)n1]

as well as all pharmaceutically acceptable salts thereof. In some embodiments, gemcitabine is gemcitabine hydrochloride.

### Combination Treatment

In an aspect is provided a method of treating a cancer in a subject. The method includes administering to the subject in need thereof a therapeutically effective amount of camonsertib and therapeutically effective amount of gemcitabine, where the therapeutically effective amount of gemcitabine is a subtherapeutic regimen of gemcitabine.

### Dosing Schedule

The ATR inhibitor (e.g., camonsertib) and the antimetabolite (e.g., gemcitabine) may be administered in combination either simultaneously (e.g., as a mixture), separately but simultaneously (e.g., via separate intravenous lines, or via multiple oral formulations, or a mixture of administration modes) or sequentially (e.g., one agent is administered first followed by administration of the second agent). Thus, the term combination is used to refer to concomitant, simultaneous or sequential administration of the ATR inhibitor (e.g., camonsertib) and the antimetabolite (e.g., gemcitabine).

In embodiments, camonsertib and gemcitabine are administered simultaneously or sequentially. In embodiments, camonsertib and gemcitabine are administered simultaneously. In embodiments, camonsertib and gemcitabine are administered sequentially. During the course of treatment camonsertib and gemcitabine may at times be administered sequentially and at other times be administered simultaneously.

In embodiments, where camonsertib and gemcitabine are administered sequentially, camonsertib is administered at a first time point and gemcitabine is administered at a second time point, where the first time point precedes the second time point. Alternatively, in embodiments, where gemcitabine and camonsertib are administered sequentially, gemcitabine is administered at a first time point and camonsertib is administered at a second time point, where the first time point precedes the second time point.

The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

The dosage of the ATR inhibitor (e.g., camonsertib) and the antimetabolite (e.g., gemcitabine) used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The ATR inhibitor (e.g., camonsertib) and the antimetabolite (e.g., gemcitabine) used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

In instances where camonsertib and gemcitabine are administered simultaneously, the camonsertib and gemcitabine may be administered as a mixture. Thus, in embodiments, camonsertib and gemcitabine are admixed prior to administration.

In instances where camonsertib and gemcitabine are administered sequentially, the time between administration of camonsertib and gemcitabine may be up to 2 weeks. In some embodiments, camonsertib is administered before gemcitabine (e.g., within 2 weeks, within 1 week, within 6 days, within 5 days, within 4 days, within 3 days, within 2 days, within 1 day, or within 12 hours). In some embodiments, camonsertib is administered after gemcitabine (e.g., within 2 weeks, within 1 week, within 6 days, within 5 days, within 4 days, within 3 days, within 2 days, within 1 day, or within 12 hours).

In some embodiments, camonsertib is administered intermittently (e.g., 1 day/week, 2 days/week, or 3 days/week). In some embodiments, gemcitabine is administered intermittently (e.g., 1 day/week, 2 days/week, or 3 days/week).

Camonsertib may be administered to the subject in a single dose or in multiple doses. Similarly, gemcitabine may be administered to the subject in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

In some embodiments, camonsertib and gemcitabine are administered to the subject in a treatment cycle. The cycle includes administration of gemcitabine to the subject in an amount of 50 to 100 mg/kg once a week, and administration of camonsertib to the subject in an amount of 5 to 30 mg/kg for three days followed by a period of four days without administration of camonsertib.

In some embodiments, the treatment cycle is repeated over a period of 28 days.

In some embodiments, camonsertib and gemcitabine are administered to the subject in a 28 day cycle. The cycle includes administration of gemcitabine to the subject in an amount of 50 to 200 mg/m² on days 1 and 15, and administration of camonsertib to the subject in an amount of 5 to 200 mg on days 1, 2, 15 and 16.

In some embodiments, camonsertib and gemcitabine are administered to the subject in a 28 day treatment cycle including administration of gemcitabine to the subject in an amount of up to 400 mg/m² on days 1 and 14, and administration of camonsertib to the subject in an amount of up to 80 mg on days 1, 2, 3, 15, 16 and 17.

In some embodiments, the treatment cycle is repeated 1, 2, 3, 4, 5, 6, 7, 8, 9, 10, 11, or 12 times.

### Dosing Amounts

In some embodiments, the subtherapeutic regimen of gemcitabine includes a starting dosage that is at least 50% less than the lowest standard starting dosage that is used for a monotherapy. In some embodiments, the subtherapeutic regimen of gemcitabine includes a maintenance dosage that is at least 50% less than the lowest standard maintenance dosage that is used for a monotherapy. In some embodiments, the maintenance dosage of gemcitabine includes a first reduced dosage. In some embodiments, the maintenance dosage of gemcitabine comprises a second reduced dosage. In some embodiments, the maintenance dosage of gemcitabine comprises a third reduced dosage.

In some instances, the subtherapeutic regimen of gemcitabine or a pharmaceutically acceptable salt thereof may be, e.g., 900 mg/m² or less (e.g., 900, 850, 800, 750, 700, 650, 600, 550, 500, 450, 400, 350, 300, 250, 200, 190, 180, 170, 160, 150, 140, 130, 120, 110, 100, 90, 80, 70, 60, 50, 40, 30, 20, or 12 mg/m² or less; e.g., 12-900, 12-850, 12-800, 12-750, 12-700, 12-650, 12-600, 12-550, 12-500, 12-450, 12-400, 12-350, 12-300, 12-250, 12-200, 12-190, 12-180, 12-170, 12-160, 12-150, 12-140, 12-130, 12-120, 12-110, 12-100, 12-90, 12-80, 12-70,12-60, 12-50,12-40, 12-30, or 12-20 mg/m²). A first reduced dosage in the subtherapeutic regimen of gemcitabine or a pharmaceutically acceptable salt thereof may be, e.g., 850 mg/day or less (e.g., 850, 800, 750, 700, 650, 600, 550, 500, 450, 400, 350, 300, 250, 200, 190, 180, 170, 160, 150, 140, 130, 120, 110, 100, 90, 80, 70, 60, 50, 40, 30, 20, or 12 mg/m² or less; e.g., 10-850, 10-800, 10-750, 10-700, 10-650, 10-600, 10-550, 10-500, 10-450, 10-400, 10-350, 10-300, 10-250, 10-200, 10-190, 10-180, 10-170, 10-160, 10-150, 10-140, 10-130, 10-120, 10-110, 10-100, 10-90, 10-80, 10-70, 10-60,10-50, 10-40, 10-30, or 10-20 mg/day). A second reduced dosage in the subtherapeutic regimen of gemcitabine or a pharmaceutically acceptable salt thereof may be, e.g., 700 mg/day or less (e.g., 700, 650, 600, 550, 500, 450, 400, 350, 300, 250, 200, 190, 180, 170, 160, 150, 140, 130, 120, 110, 100, 90, 80, 70, 60, 50, 40, 30, 20, or 12 mg/m² or less; e.g., 8-700, 8-650, 8-600, 8-550, 8-500, 8-450, 8-400, 8-350, 8-300, 8-250, 8-200, 8-190, 8-180, 8-170, 8-160, 8-150, 8-140, 8-130, 8-120, 8-110, 8-100, 8-90, 8-80, 8-70, 8-60, 8-50, 8-40, 8-30, 8-20, or 8-10 mg/m²). A third reduced dosage in the subtherapeutic regimen of gemcitabine or a pharmaceutically acceptable salt thereof may be, e.g., 400 mg/m² or less (e.g., 400, 350, 300, 250, 200, 190, 180, 170, 160, 150, 140, 130, 120, 110, 100, 90, 80, 70, 60, 50, 40, 30, 20, or 12 mg/m² or less; e.g., 6-400, 6-350, 6-300, 6-250, 6-200, 6-190, 6-180, 6-170, 6-160, 6-150, 6-140, 6-130, 6-120, 6-110, 6-100, 6-90, 6-80, 6-70, 6-60, 6-50, 6-40, 6-30, 6-20, or 6-10 mg/m²).

In some embodiments, gemcitabine is administered in a dosage ranging from 10 mg/m² to 400 mg/m². For example, gemcitabine can be agminated in a dosage of 10 mg/m², 25 mg/m², 50 mg/m², 75 mg/m², 100 mg/m², 125 mg/m², 150 mg/m², 175 mg/m², 200 mg/m², 250 mg/m², 275 mg/m², 300 mg/m², 325 mg/m², 350 mg/m², 375 mg/m², or 400 mg/m².

In some embodiments, gemcitabine is administered in a dosage of 100 mg/m² or 200 mg/m².

In some embodiments, the dosage of gemcitabine is 100 mg/m².

In some embodiments, the dosage of gemcitabine is 200 mg/m².

In embodiments, gemcitabine is administered in an amount of 50 mg/kg or 100 mg/kg.

In embodiments, gemcitabine is administered at an amount of about 100 mg/kg, 90 mg/kg, 80 mg/kg, 70 mg/kg, 60 mg/kg, 50 mg/kg, 40 mg/kg, 30 mg/kg, 20 mg/kg, 10 mg/kg or 5 mg/kg. In embodiments, gemcitabine is administered at an amount of about 100 mg/kg. In embodiments, gemcitabine is administered at an amount of about 90 mg/kg. In embodiments, gemcitabine is administered at an amount of about 80 mg/kg. In embodiments, gemcitabine is administered at an amount of about 70 mg/kg. In embodiments, gemcitabine is administered at an amount of about 60 mg/kg. In embodiments, gemcitabine is administered at an amount of about 50 mg/kg. In embodiments, gemcitabine is administered at an amount of about 40 mg/kg. In embodiments, gemcitabine is administered at an amount of about 30 mg/kg. In embodiments, gemcitabine is administered at an amount of about 20 mg/kg. In embodiments, gemcitabine is administered at an amount of about 10 mg/kg. In embodiments, gemcitabine is administered at an amount of about 5 mg/kg.

Preferably, the dosage of gemcitabine is a low dosage (e.g., at least 10%, 20%, 50%, 80%, 90%, or 95% less than the lowest standard recommended dosage of gemcitabine for a given route of administration). The standard recommended dose of gemcitabine for differing indications in provided in Table 1 below (from gemcitabine label). Preferably, gemcitabine is administered once daily or twice daily.

TABLE 1
Standard Recommended Doses of Gemcitabine

| | Standard | |
|---|---|---|
| | gemcitabine | |
| Indication | dose | Dose reduction guideline |
| Pancreatic | 1000 mg/m² | 75% of dose or hold |
| cancer | | depending on lab value |
| (single agent) | | |
| Non-small cell | 1250 mg/m² | 75% of dose or hold |
| lung cancer (in | | depending on lab value |
| combination | | |
| with cisplatin) | | |
| Breast cancer | 1250 mg/m² | 75% or 50% (625 mg/m²) of dose or |
| (in combination | | hold drug depending on lab value |
| with paclitaxel) | | |
| Ovarian cancer | 1000 mg/m² | For cycle after event of myelosuppresion: |
| (in combination | | Reduce dose to 800 mg/m² on days 1, 8; |
| with | | subsequent occurrence reduce to |
| carboplatin) | | 800 mg/m², dose only on day 1 |

In some embodiments, the therapeutically effective amount of camonsertib is a subtherapeutic regimen of camonsertib.

In some embodiments, the subtherapeutic regimen of camonsertib includes a starting dosage that is at least 50% less than the lowest standard starting dosage that is used for a monotherapy. In some embodiments, the subtherapeutic regimen of camonsertib includes a maintenance dosage that is at least 50% less than the lowest standard maintenance dosage that is used for a monotherapy. In some embodiments, the maintenance dosage of camonsertib includes a first reduced dosage. In some embodiments, the maintenance dosage of camonsertib comprises a second reduced dosage. In some embodiments, the maintenance dosage of camonsertib comprises a third reduced dosage.

Preferably, the subtherapeutic regimen of camonsertib is a low dosage (e.g., at least 10%, 20%, 50%, 80%, 90%, or 95% less than the lowest standard recommended dosage of camonsertib for a given route of administration). Preferably, camonsertib is administered once daily or twice daily.

In some instances, a starting dosage in the subtherapeutic regimen of camonsertib or a pharmaceutically acceptable salt thereof may be, e.g., 160 mg/day or less (e.g., 160, 150, 140, 130, 120, 110, 100, 90, 80, 70, 60, 50, 40, 30, 20, 10, 5, or 3 mg/day or less; e.g., 3-160, 3-150, 3-140, 3-130, 3-120, 3-110, 3-100, 3-90, 3-80, 3-70, 3-60, 3-50, 3-40, 3-30, 3-20, 3-10, or 3-5 mg/day). A first reduced dosage in the subtherapeutic regimen of camonsertib or a pharmaceutically acceptable salt thereof may be, e.g., 100 mg/day or less (e.g., 100, 90, 80, 70, 60, 50, 40, 30, 20, 10, 5, or 3 mg/day or less; e.g., 2-100, 2-90, 2-80, 2-70, 2-60, 2-50, 2-40, 2-30, 2-20, 2-10, or 2-5 mg/day). A second reduced dosage in the subtherapeutic regimen of camonsertib or a pharmaceutically acceptable salt thereof may be, e.g., 95 mg/day or less (e.g., 90, 85, 80, 75, 70, 65, 60, 55, 50, 45, 40, 35, 30, 25, 20, 15, 10, 5, or 3 mg/day or less; e.g., 1-95, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, or 1-3 mg/day). A third reduced dosage in the subtherapeutic regimen of camonsertib or a pharmaceutically acceptable salt thereof may be, e.g., 80 mg/day or less (e.g., 80, 75, 70, 65, 60, 55, 50, 45, 40, 35, 30, 25, 20, 15, 10, 5, or 3 mg/day or less; e.g., 1-80, 1-75, 1-70, 1-65, 1-60, 1-55, 1-50, 1-45, 1-40, 1-35, 1-30, 1-25, 1-20, 1-15, 1-10, 1-5, or 1-3 mg/day).

In embodiments, camonsertib is administered in an amount ranging from 10 mg to 200 mg. In embodiments, camonsertib is administered in an amount of about 10 mg. In embodiments, camonsertib is administered in an amount of about 20 mg. In embodiments, camonsertib is administered in an amount of about 30 mg. In embodiments, camonsertib is administered in an amount of about 40 mg. In embodiments, camonsertib is administered in an amount of about 50 mg. In embodiments, camonsertib is administered in an amount of about 60 mg. In embodiments, camonsertib is administered in an amount of about 70 mg. In embodiments, camonsertib is administered in an amount of about 80 mg. In embodiments, camonsertib is administered in an amount of about 90 mg. In embodiments, camonsertib is administered in an amount of about 100 mg. In embodiments, camonsertib is administered in an amount of about 110 mg. In embodiments, camonsertib is administered in an amount of about 120 mg. In embodiments, camonsertib is administered in an amount of about 130 mg. In embodiments, camonsertib is administered in an amount of about 140 mg. In embodiments, camonsertib is administered in an amount of about 150 mg. In embodiments, camonsertib is administered in an amount of about 160 mg. In embodiments, camonsertib is administered in an amount of about 170 mg. In embodiments, camonsertib is administered in an amount of about 180 mg. In embodiments, camonsertib is administered in an amount of about 190 mg. In embodiments, camonsertib is administered in an amount of about 200 mg.

In embodiments, camonsertib is administered in an amount of 80 mg or 120 mg.

In embodiments, camonsertib is administered at an amount of about 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In embodiments, camonsertib is administered at an amount of about 30 mg/kg. In embodiments, camonsertib is administered at an amount of about 20 mg/kg. In embodiments, camonsertib is administered at an amount of about 10 mg/kg. In embodiments, camonsertib is administered at an amount of about 9 mg/kg. In embodiments, camonsertib is administered at an amount of about 8 mg/kg. In embodiments, camonsertib is administered at an amount of about 7 mg/kg. In embodiments, camonsertib is administered at an amount of about 6 mg/kg. In embodiments, camonsertib is administered at an amount of about 5 mg/kg. In embodiments, camonsertib is administered at an amount of about 4 mg/kg. In embodiments, camonsertib is administered at an amount of about 3 mg/kg. In embodiments, camonsertib is administered at an amount of about 2 mg/kg. In embodiments, camonsertib is administered at an amount of about 1 mg/kg.

In some embodiments, camonsertib is administered in amount of 5 mg/kg, 10 mg/kg or 30 mg/kg.

### Indications

The methods of the present application can be used for the treatment of a disease or condition having the symptom of cell hyperproliferation. For example, the method described herein may be applicable for treatment of various oncological conditions harboring sensitizing gene mutations, such as tumors with any deleterious (loss-of-function) DNA damage response alterations. In some embodiments, the disease to be treated is cancer.

In some embodiments, the cancer is associated with an abnormal ATR activity. The cancer can be associated with an ATRi-sensitizing gene alteration, including a loss of function of ATM, ATRIP, BRCA1, BRCA2, CDK12, CHTF8, FZR1, MRE11, NBN, PALB2, RAD17, RAD50, RAD51B/C/D, REV3L, RNASEH2A, RNASEH2B or SETD2.

In some embodiments, the loss of function is a loss of function of ATM. In some embodiments, the loss of function is a loss of function of ATRIP. In some embodiments, the loss of function is a loss of function of BRCA1. In some embodiments, the loss of function is a loss of function of BRCA2. In some embodiments, the loss of function is a loss of function of CDK12. In some embodiments, the loss of function is a loss of function of CHTF8. In some embodiments, the loss of function is a loss of function of FZR1. In some embodiments, the loss of function is a loss of function of MRE11. In some embodiments, the loss of function is a loss of function of NBN. In some embodiments, the loss of function is a loss of function of PALB2. In some embodiments, the loss of function is a loss of function of RAD17. In some embodiments, the loss of function is a loss of function of RAD50. In some embodiments, the loss of function is a loss of function of RAD51B/C/D. In some embodiments, the loss of function is a loss of function of REV3L. In some embodiments, the loss of function is a loss of function of RNASEH2A. In some embodiments, the loss of function is a loss of function of RNASEH2B. In some embodiments, the loss of function is a loss of function of SETD2.

In particular, mutations in one or more of these genes may be frequently found in the following tumor types: renal cell carcinoma, mature B-cell neoplasms, endometrial cancer, ovarian cancer, colorectal cancer, skin cancer (non-melanoma), small bowel cancer, non-small cell lung cancer, melanoma, bladder cancer, pancreatic cancer, head and neck cancer, mesothelioma, glioma, prostate cancer, breast cancer, and esophagogastric cancer. Accordingly, methods of the present application are preferably used in the treatment of these cancers.

The disease or condition treated using methods of the invention may have the symptom of cell hyperproliferation. For example, the disease or condition may be a cancer. The cancer may be, e.g., carcinoma, sarcoma, adenocarcinoma, lymphoma, leukemia, or melanoma. The cancer may be, e.g., a solid tumor.

Non-limiting examples of cancers include prostate cancer, breast cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, pancreatic cancer, kidney cancer, bladder cancer, colon cancer, and liver cancer.

The methods of the present application can be used in the treatment of renal cell carcinoma, mature B-cell neoplasms, endometrial cancer, ovarian cancer, fallopian tube cancer, primary peritoneal cancer, colorectal cancer, skin cancer (non-melanoma), small bowel cancer, non-small cell lung cancer, melanoma, bladder cancer, pancreatic cancer, head and neck cancer, mesothelioma, glioma, prostate cancer, breast cancer, or esophagogastric cancer.

Preferably, the methods of the present application can be used in the treatment of cancer associated with a loss of function of ATM, BRCA1, BRCA2, or PALB2. The methods disclosed herein are preferably used in the treatment of ovarian, pancreatic, prostate, or lung cancer.

Non-limiting examples of carcinomas include medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Non-limiting examples of sarcomas include chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Non-limiting examples of leukemias include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

Non-limiting examples of melanomas include acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

### Subjects

The methods of the present application can be carried out, for example, with subjects who have had no or only a limited amount of prior treatment (for example, one, two, or three treatment regimens involving surgery, chemotherapy and/or biological therapy) and in patients with locally advanced or metastatic cancer.

In the case of subjects selected on the basis of prior treatment, the methods of the invention include the treatment of patients who have not received any prior treatment regimen (for example, a treatment regimen involving chemotherapy and/or biological therapy). In these patients, treatment according to the methods of the invention can be called, in various examples, a “first line” treatment.

In some embodiments, the methods of the application may be used with subjects who have received a single prior regimen of treatment (for example, treatment involving chemotherapy and/or biological therapy), in which case treatment according to the methods of the present application can be called, in various examples, a “second line” treatment. These patients typically have been treated previously with a single regimen involving administration of, for example, an antibody (e.g., trastuzumab), a hormonal agent, capecitabine, an anthracycline (e.g., doxorubicin, epirubicin, daunorubicin, or idarubicin), a taxane (e.g., paclitaxel or docetaxel), a platinum (e.g., cisplatin, or carboplatin), or a combination thereof. In other embodiments, the methods of the invention may be used with patients that have had no more than two prior treatment regimens. In other embodiments, the methods of the invention may be used with subjects that have had two or more prior treatment regimens (and can be called, in various examples, “third line”).

As is understood in the art, a treatment regimen in cancer therapy does not typically involve administration of a single dose of a drug. Rather, a treatment regimen involves multiple cycles of drug administration that are typically designed so that a patient has the opportunity to recover from side effects of the drug between the cycles. Thus, for example, a patient who has received a single prior treatment regimen of a drug may have received the drug, for example, in 3-8 different doses separated from one another by 1-2 weeks. Such an administration regimen, or a substantial portion thereof (e.g., at least half of the regimen), can be considered as a single prior treatment regimen in the selection of patients to treat with the combination of the present application as a second-line treatment.

In some embodiments, the combined treatment is an adjuvant treatment combined with surgical removal or partial surgical removal of the cancer. The methods disclosed herein may be utilized for the purpose of reducing tumor size before surgical operation (referred to as preoperative adjuvant chemotherapy or neoadjuvant therapy), or may be utilized for the purpose of preventing recurrence of tumor after surgical operation (referred to as postoperative adjuvant chemotherapy or adjuvant therapy).

### Synergy

In embodiments, the ATR inhibitor (e.g., camonsertib) and the antimetabolite (e.g., gemcitabine) are administered in a combined synergistic amount. In embodiments, camonsertib and gemcitabine are administered in a combined synergistic amount. A “combined synergistic amount” as used herein refers to the sum of a first amount (e.g., an amount of camonsertib) and a second amount (e.g., an amount of gemcitabine) that results in a synergistic effect (i.e., an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of gemcitabine when used separately from camonsertib. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of camonsertib when used separately from gemcitabine.

The synergistic effect may be a cancer-treating effect such as a lymphoma (i.e., a lymphoma-treating synergistic effect), leukemia (i.e., a leukemia-treating synergistic effect), myeloma (i.e., a myeloma-treating synergistic effect), carcinoma (i.e., a carcinoma-treating synergistic effect), mature B-cell neoplasms (i.e., a mature B-cell neoplasm-treating synergistic effect), endometrial cancer (i.e., an endometrial cancer-treating synergistic effect), ovarian cancer (i.e., an ovarian-treating synergistic effect), fallopian tube cancer (i.e., a fallopian tube cancer-treating synergistic effect), primary peritoneal cancer (i.e., a primary peritoneal cancer-treating synergistic effect), colorectal cancer (i.e., a colorectal cancer-treating synergistic effect), skin cancer (i.e., a skin cancer-treating synergistic effect), small bowel cancer (i.e., a small bowel cancer-treating synergistic effect), non-small cell lung cancer (i.e., a non-small cell lung cancer-treating synergistic effect), melanoma (i.e., a melanoma-treating synergistic effect), bladder cancer (i.e., a bladder cancer-treating synergistic effect), pancreatic cancer (i.e., a pancreatic cancer-treating synergistic effect), head and neck cancer (i.e., a head and neck-treating synergistic effect), mesothelioma (i.e., a mesothelioma-treating synergistic effect), glioma (i.e., a glioma-treating synergistic effect), prostate cancer (i.e., a prostate cancer-treating synergistic effect), breast cancer (i.e., a breast cancer-treating synergistic effect), or esophagogastric cancer (i.e., a esophagogastric cancer-treating synergistic effect), treating effect.

### Toxicity

A possible side effect of the treatment with subjects with anticancer agents is neutropenia, which is characterized by a reduced number of neutrophils. Unfortunately, a number of neutropenia deaths have been reported among subjects treated with anti-cancer agents.

A surprising result of the method of the present invention is a reduction in treatment emergent adverse event, even with repeated treatment (e.g., a reduction in neutropenia, fatigue, nausea, etc.). In some embodiments, the administering results in a reduction of neutrophils of less than 15%. In some embodiments, the administering results in a reduction of neutrophils of less than 14%. In some embodiments, the administering results in a reduction of neutrophils of less than 13%. In some embodiments, the administering results in a reduction of neutrophils of less than 12%. In some embodiments, the administering results in a reduction of neutrophils of less than 11%. In some embodiments, the administering results in a reduction of neutrophils of less than 10%. In some embodiments, the administering results in a reduction of neutrophils of less than 9%. In some embodiments, the administering results in a reduction of neutrophils of less than 8%. In some embodiments, the administering results in a reduction of neutrophils of less than 7%. In some embodiments, the administering results in a reduction of neutrophils of less than 6%. In some embodiments, the administering results in a reduction of neutrophils of less than 5%.

The methods of the present application can include a step of monitoring the subjects blood counts, and measuring neutrophil levels. In one embodiment, the monitoring comprises taking a blood sample from the subject. The blood sample is analyzed for neutrophil count.

Determining neutrophil counts can be performed according to procedures well known to those skilled in the art.

One aspect of the present application is a method of reducing the risk of neutropenia in a subject being treated for cancer. The method includes administering to the subject in need thereof a therapeutically effective amount of camonsertib and therapeutically effective amount of gemcitabine, where the therapeutically effective amount of gemcitabine is a subtherapeutic regimen of gemcitabine. A blood sample is obtained from the subject and neutrophil levels measured. If the neutrophil count has decreased below 1,500 cells/mm³, the amount of gemcitabine is reduced. Alternatively, the amount of camonsertib can be reduced, and or the amounts of both camonsertib and gemcitabine are reduced.

### Route of Administration

The compounds identified as capable of treating any of the conditions described herein, using any of the methods described herein, may be administered to patients or animals with a pharmaceutically acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to subjects in need thereof. Administration may begin before the patient is symptomatic.

Exemplary routes of administration of the compounds (e.g., camonsertib and gemcitabine), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention.

In some embodiments, the combination treatment utilizes multiple routes of administration (e.g., via intraventricular and oral).

### Pharmaceutical Compositions

The camonsertib and gemcitabine used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The camonsertib and gemcitabine can also be used in the form of the free bases, in the form of salts, zwitterions, solvates, or as prodrugs, or pharmaceutical compositions thereof. All forms are within the scope of the present application. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., 40 mesh.

### Formulations for Oral Administration

The camonsertib and/or gemcitabine can be formulated into a composition for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution- or diffusion- controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The camonsertib and gemcitabine for use in the present methods can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: In Vitro Synergy Between Camonsertib and Gemcitabine

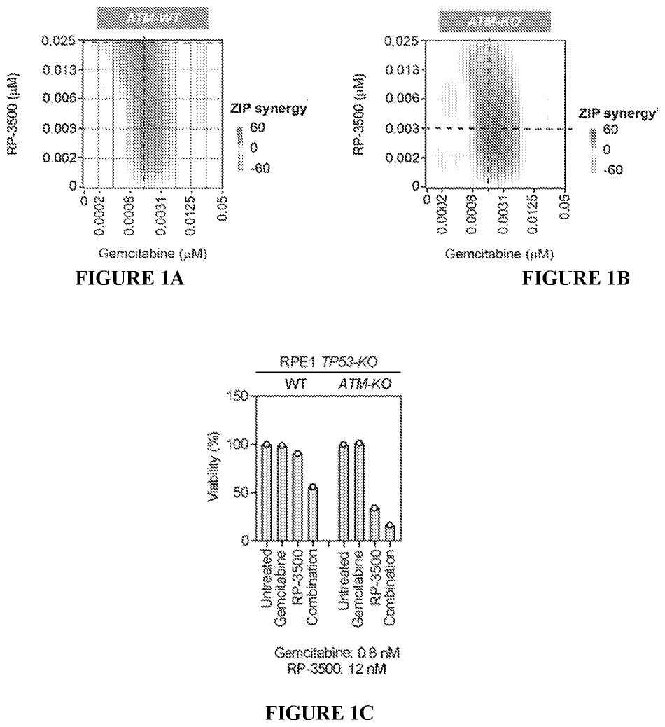

To determine the degree of synergy between camonsertib and gemcitabine, a Zero Interaction Potency (ZIP) analysis was carried out using isogenic TP53-deficient RPE1 cells that were either ATM wild type (WT) or ATM knockout (KO). In this analysis, a ZIP score of greater than 10 indicates synergy whereas a score of less than 10 indicates antagonism. Camonsertib and gemcitabine strongly synergized in both ATM-WT and ATM-KO cells (FIGS. 1A-1C). Importantly, in ATM-KO cells, the strongest synergy was observed at markedly lower concentrations of camonsertib, leading to strong cytotoxicity at doses that only modestly affected ATM-WT cells (FIGS. 1A-C). These results show that tumor cells carrying ATRi-sensitizing genetic alterations, such as ATM are hypersensitive to a camonsertib+gemcitabine combination.

### Example 2: Low Dose Gemcitabine Synergy with Camonsertib in an In Vitro BRCA1mut Model

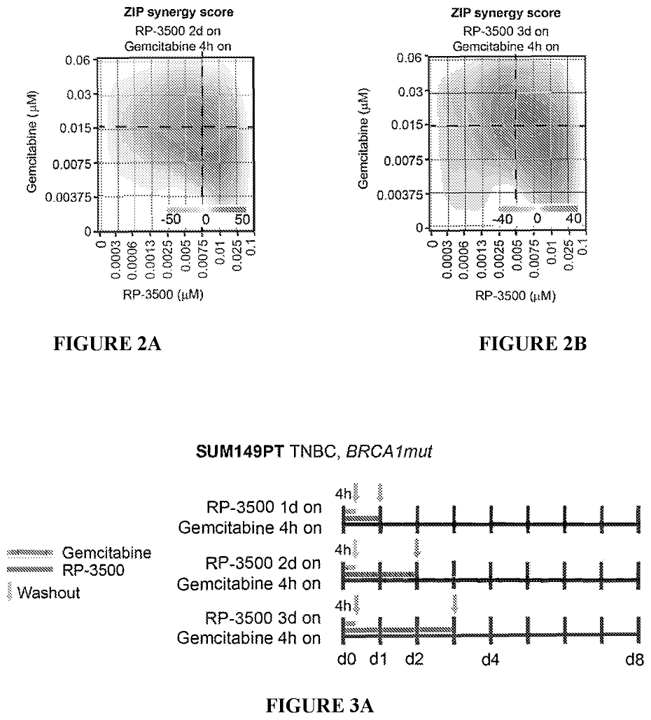

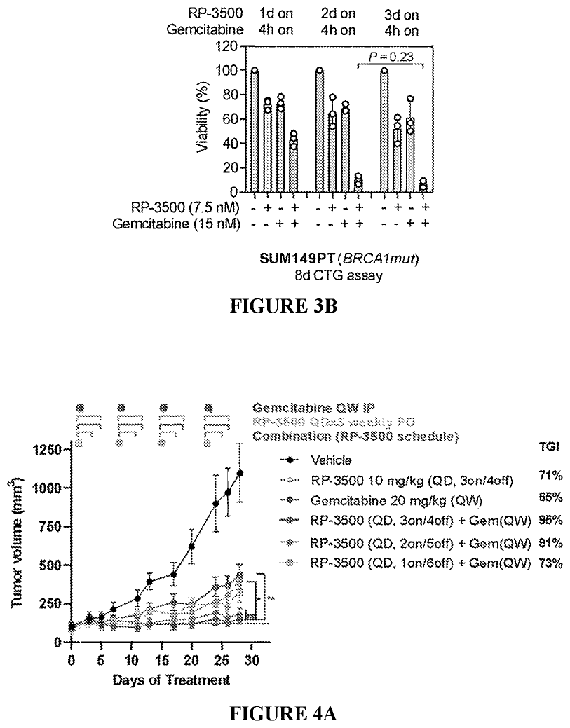

To assess the synergy between camonsertib and gemcitabine in a BRCA1-mutated cancer cell model in vitro, a SUM149PT breast cancer cell line was employed. The combination was administered at various dosing schedules—1, 2, or 3 day treatment with camonsertib combined with a single 4 h dose of gemcitabine (FIG. 3A). Camonsertib strongly synergized with markedly low doses of gemcitabine (low nM) using both 2 d and 3 d treatment (FIGS. 2A and 2B; note that therapeutic gemcitabine doses in humans leads to mean gemcitabine peak plasma concentrations ranging from 24 μM at 800 mg/m² to 32 μM at 1000 mg/m² (van Moorsel C J et al., Ann Oncol. 10:441-448 (1999); Kroep J P et al., J Clin OncoL 17:2190-2197 (1999))).

As a result, the combination of camonsertib and gemcitabine killed SUM149PT cells at low nM concentrations, despite these doses showing only modest effect as single agent (FIG. 3B). 2 d treatment was as effective as 3 d treatment (FIG. 3B). These data suggest that BRCA1/2-mutated cancer cells are hypersensitive to a camonsertib+gemcitabine combination and that administration of the combination is sufficient to exert tumor cytotoxicity in vitro.

### Example 3: In Vivo Anti-Tumor Efficacy of Camonsertib in Combination with Gemcitabine in a SUM149PT BRCA1 Mutated Xenograft Model

A xenograft model was employed to assess the effect of camonsertib in combination with gemcitabine on tumor formation in an animal model. Mice harboring established tumors were treated with a vehicle control, camonsertib (10 mg/kg once per day with 3 days on/4 days off), gemcitabine (20 mg/kg once per week), or a combination of both camonsertib (once per day selected from 3 days on/4 days off, 2 days on/5 days off, or 1 day on/6 days off) and gemcitabine (once per week) for 28 days.

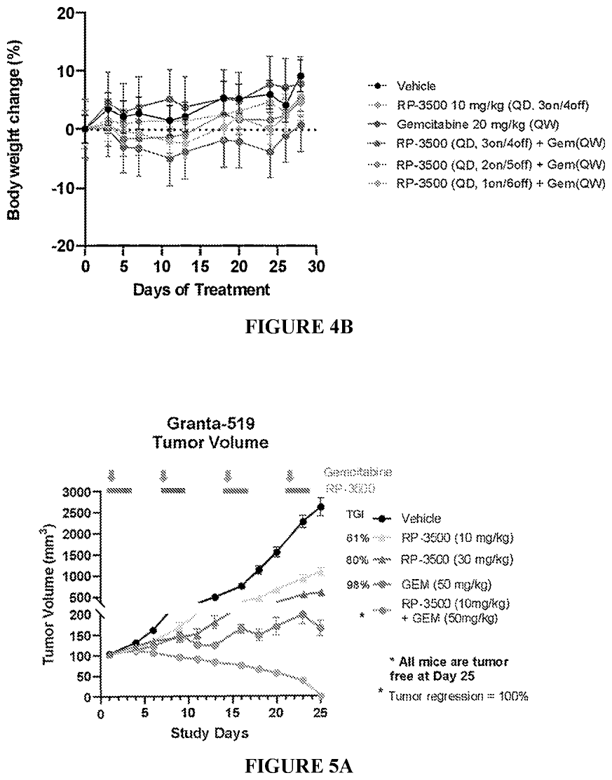

Relative tumor xenograft volume of the mice treated with gemcitabine at 20 mg/kg QW on Day 1 and/or camonsertib at 10 mg/kg PO on different schedules weekly for 28 days is shown in FIG. 4A and the change in body weight is shown in FIG. 4B. Results are expressed as mean±SEM, N=7/group in female NOD-SCID mice. Statistical differences were established by Student's T-test (GraphPad Prism v9) with Welch's correction; ns=not significant, *p<0.05, **p<0.01. The results indicate a reduced activity with low doses of the single agents (camonsertib or gemcitabine). Improved cytotoxic activity was obtained with camonsertib combined with a low dose of gemcitabine (gemcitabine at 20% of maximum tolerated dose in mice (MTD)). Additionally, no significant impact on body weight was observed. This data shows that weekly intermittent dosing of camonsertib together with once-weekly low dose gemcitabine is more efficacious than each single agent (FIGS. 4A-4B).

### Example 4: In Vivo Anti-Tumor Efficacy of Camonsertib in Combination with Gemcitabine in a Granta-519 (ATM Deficient) Mantle Cell Lymphoma Xenograft Model

A xenograft model was employed to assess the effect of camonsertib in combination with gemcitabine on tumor formation in mice harboring Granta-519 (ATM deficient) mantle cell lymphoma. The mice were treated with a vehicle control, gemcitabine at 50 or 100 mg/kg QW on Day 1 and/or camonsertib at 5, 10 or 30 mg/kg PO 3 days on/4 days off weekly for 28 days.

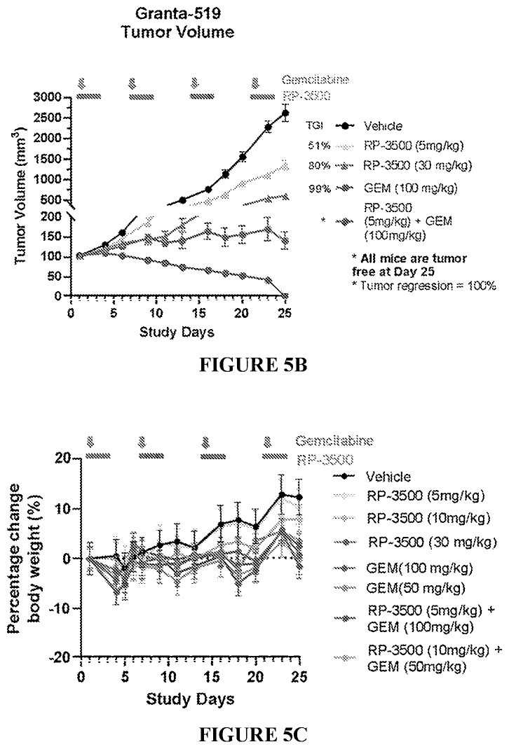

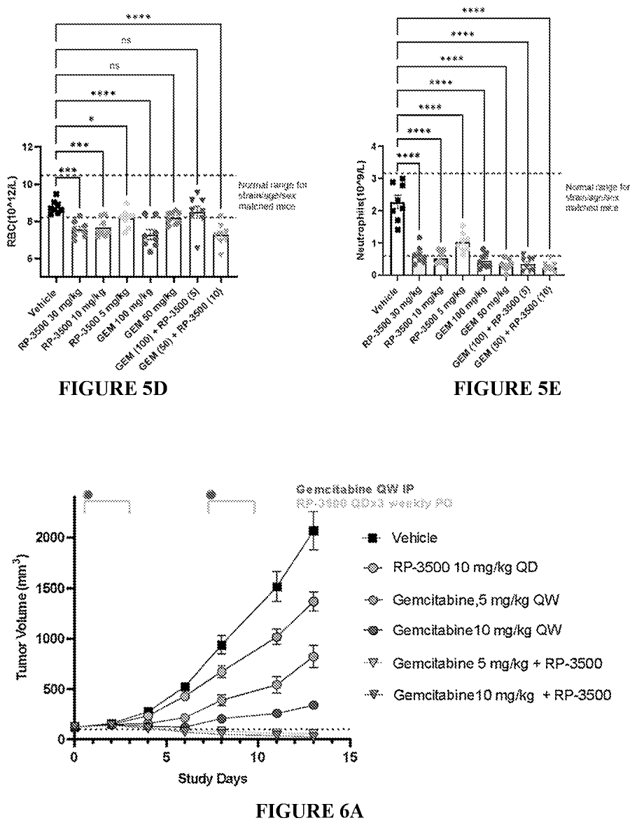

Relative tumor xenograft volumes of the mice are shown in FIGS. 5A-5B and the change in body weight is shown in FIG. 5C. The results are expressed as mean±SEM, N=8/group in female NOD-SCID mice. The results show complete tumor regression observed in all mice by Day 25 treated with camonsertib and the gemcitabine combination.

FIGS. 5D and 5E show the red blood cell and neutrophil count, respectively, for the mice on day 28. The combination treatment appears to increase anemia with increasing camonsertib doses. Neutropenia in the combination treatment increases with gemcitabine and camonsertib dose. A decrease in monocytes was also observed in the combination groups (data not shown). However, no meaningful differences in platelets were observed in any combination (data not shown).

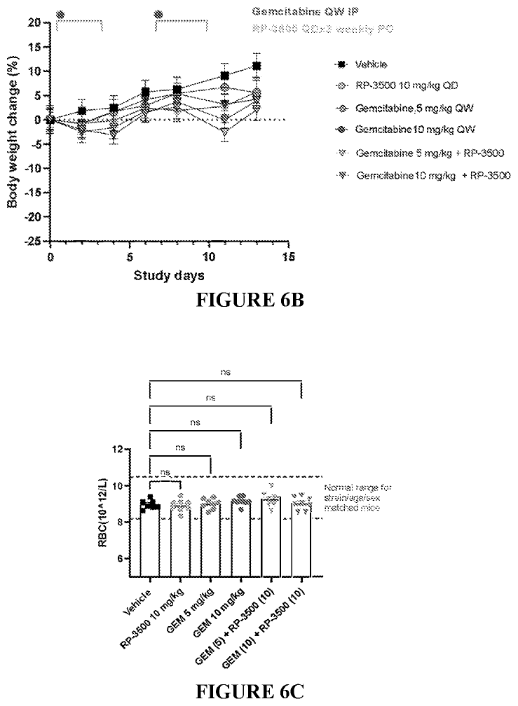

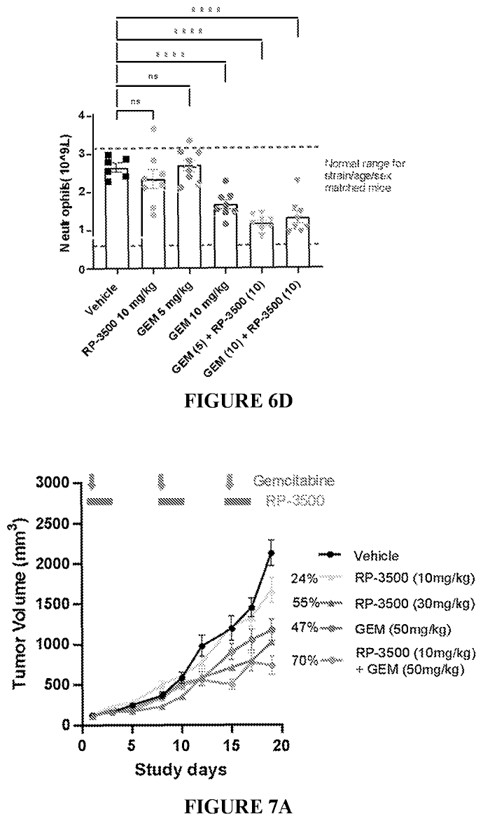

A second study in the same model was conducted with reduced doses of both gemcitabine and camonsertib. Mice were treated with vehicle control, gemcitabine at 5 or 10 mg/kg QW on Day 1 and/or camonsertib at 10 mg/kg QD for two weeks (N=8 per group). Plots of the tumor volume and body weight are shown in FIGS. 6A and 6B, respectively. Hematology parameters were measured on day 15. No effect on red blood cells (Day 15) was observed (FIG. 6C). A decrease in neutrophils was observed in the combination groups despite minimal decrease occurring with the single agent low dose gemcitabine (FIG. 6D). The results indicate that camonsertib in combination with very low doses of gemcitabine show enhanced anti-tumor efficacy. The combination is well tolerated at these doses of camonsertib (⅓ MTD in mice) and gemcitabine ( 1/10^{th}- 1/20^{th} MTD in mice).

### Example 5: In Vivo Anti-Tumor Efficacy of Camonsertib in Combination with Gemcitabine in a Capan-1 Xenograft Model

A xenograft model was employed to assess the effect of camonsertib in combination with gemcitabine on tumor formation in mice harboring Capan-1 (BRCA2 mut) pancreatic cancer. The mice were treated with a vehicle control, gemcitabine at 50 or 100 mg/kg QW on Day 1 and/or camonsertib at 5, 10 or 30 mg/kg PO 3 days on/4 days off weekly for 20 days.

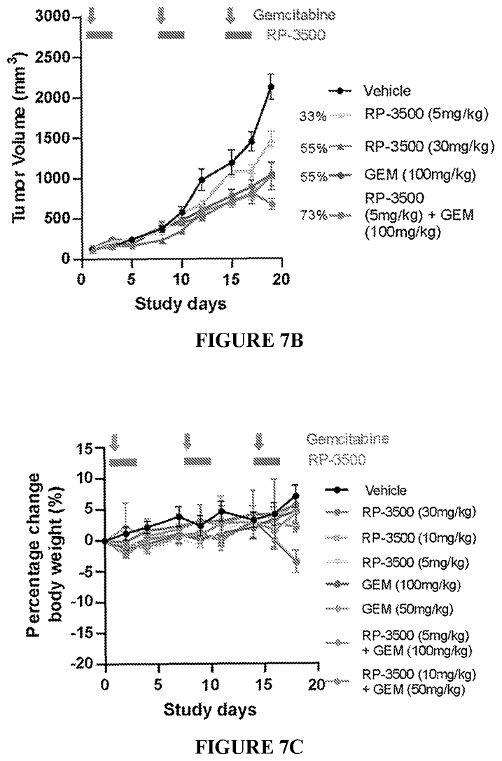

The tumor xenograft volume of the mice is shown in FIGS. 7A-7B and the change in body weight is shown in FIG. 7C. The results are expressed as mean±SEM, N=8/group. % tumor growth inhibition at day 19 is indicated to the left of the legend.

Increased efficacy for the combination treatment was observed over the single agent camonsertib therapy at MTD (30 mg/kg). The combination treatment of ½ gemcitabine MTD (50 mg/kg) and ⅓ camonsertib MTD (10 mg/kg) as well as the treatment of gemcitabine MTD (100 mg/kg) and ⅙ of camonsertib MTD (5 mg/kg) were well tolerated. No clinical observations were detected with the single agent or either dose combinations.

Applicants preclinical studies have shown that combinations of camonsertib (at ≤50% of the maximum tolerated dose [MTD] in mice) with gemcitabine (as low as 5% of the MTD in mice) exhibits synergistic antitumor activity in vivo, with tumor regression and better efficacy than either single agent at their MTD. The combination is well tolerated suggesting low doses of gemcitabine can induce replication stress and thus synergize with ATRi to augment tumor cell death.

### Example 6: Safety, Pharmacokinetics, Pharmacodynamics and Preliminary Clinical Activity of Camonsertib in Combination with Gemcitabine in Advanced Solid Tumors with ATR Inhibitor Sensitizing Mutations

The TRESR Study (Treatment Enabled by SNIPRx) is a Phase ½a study of the safety, pharmacokinetics, pharmacodynamics and preliminary clinical activity of camonsertib alone or in combination with talazoparib or gemcitabine in advanced solid tumors with ATR inhibitor sensitizing mutations. Module 4 of the TRESR study evaluates the safety and tolerability of camonsertib in combination with gemcitabine and to establish a safe and well-tolerated recommended phase 2 dose (RP2D). Notably, the TRESR study is the first to evaluate the safety and efficacy of an ATRi in combination with low doses of gemcitabine.

Module 4 of TRESR study enrolled patients (>18 years, ECOG 0-1) with advanced solid tumors with deleterious germline or somatic DDR alterations. Prior treatment with gemcitabine was permitted; given it was not the most recent prior line. Further acceptance parameters included a hemoglobin level of ≥10 g/dL, platelets ≥140 K/μL, and ANC ≥1.7 K/μL.

The primary objectives/key endpoints considered were safety and tolerability as well as a recommended phase 2 dose and schedule. The secondary objectives/key endpoints considered included: (1) overall response (RECIST v1.1, PSA, or CA-125 response), (2) clinical benefit (response or ≥16 weeks on treatment without progression), and (3) PK parameters of camonsertib in combination with gemcitabine. The exploratory objectives/key endpoints included the genomic analysis and ctDNA molecular response.

Tables 2 and 3 below show the patient demographics.

TABLE 2
Patient Demographics

| Parameter | N = 62 |
|---|---|
| Age (years) | |
| Median (IQR) | 61 (55-70) |
| Sex, n (%) | |
| Male/female | 15 (24)/47 (76) |
| ECOG PS, n (%) | |
| 0 | 25 (40) |
| 1 | 37 (60) |
| Prior lines of systemic therapy | |
| Median (IQR) | 3 (2-4) |
| ≥3, n (%) | 36 (60) |
| Prior platinum | 51 (82) |
| Prior PARP inhibitor | 34 (55) |
| Ovarian | 20 (74) |
| Prior gemcitabine | 10 (16) |

TABLE 3
Patient Cancer Demographics

| Tumor types, n (%) | N = 62 |
|---|---|
| Ovarian | 27 (44) |
| Pancreatic | 8 (13) |
| Breast | 6 (10) |
| Colorectal | 4 (6) |
| Prostate | 3 (5) |
| Lung (including NSCLC) | 3 (5) |
| Endometrial | 2 (3) |
| Liver | 2 (3) |
| Other^{a} | 7 (11) |
| Genotypes, n (%) | |
| BRCA1 | 22 (32) |
| BRCA2 | 16 (26) |
| ATM | 14 (23) |
| PALB2 | 2 (3) |
| CDK12 | 2 (3) |
| SETD2 | 2 (3) |
| Other^{b} | 4 (6) |

^{a}Cervical (n = 1), gastrointestinal (n = 1), head and neck (n = 1), kidney (n = 1), ampullary (n = 1), mesothelioma (n = 1), and uterine carcinosarcoma (n = 1).
^{b}RAD50 (n = 1), RAD51B (n = 1), RAD51C (n = 1), and MRE11A (n = 1).
ECOG PS: Eastern Cooperative Oncology Group performance status; IQR: interquartile range; PARP: poly (ADP-ribose) polymerase.

In Arm 1, patients were treated with tailored combinatorial doses of gemcitabine dose (de-escalating from a standard 1000 mg/m² dose) in combination with camonsertib starting at 80 mg QD (half the current monotherapy dose) with escalations to 120 mg QD. Gemcitabine was dosed on Days 1 and 8, while camonsertib was dosed on Days 1-3 and 8-10 of a 21-day cycle (2 weeks on/1 week off dosing schedule). Subsequent dose levels in Arm 1 evaluated decreasing gemcitabine doses (800-400 mg/m²) as well as alternative dosing schedules (camonsertib on a 2 days on/5 days off schedule; both drugs on a 1 week on/1 week off schedule [28-day cycle]).

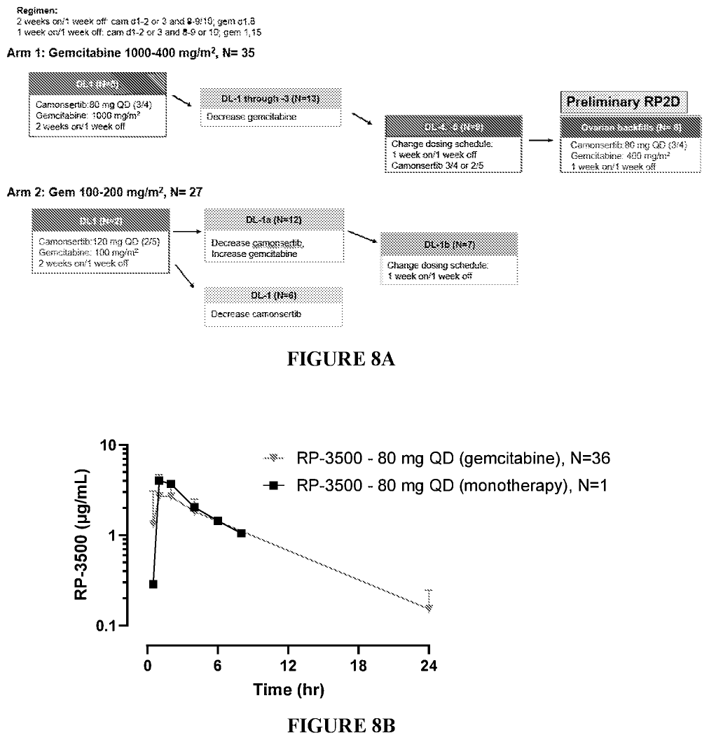

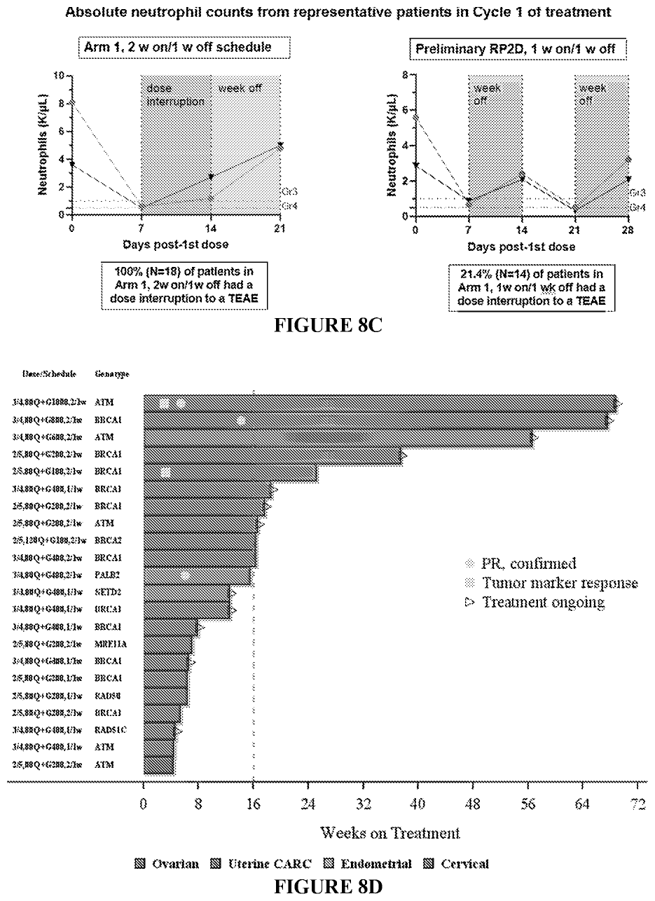

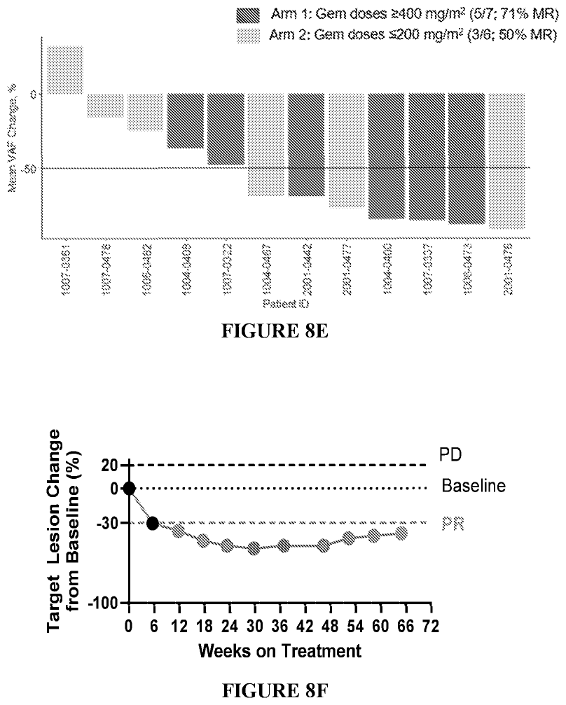

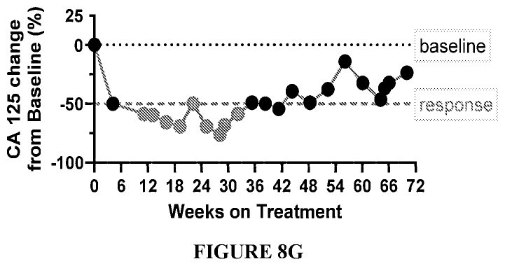

Due to the potential synergy and selective enhancement of cytotoxicity in the appropriate tumor genetic backgrounds when combining low doses of gemcitabine and camonsertib, and in order to avoid dose-limiting neutropenia, patients in Arm 2 (N=27) received 80 or 120 mg camonsertib with low dose gemcitabine (<400 mg/m²) on a 21 or 28 day (1 week on/1 week off) schedule. FIG. 8A shows the breakdown of treatment and subjects in both Arm 1 and Arm 2.

The patients were monitored to determine the camonsertib PK level when combined with gemcitabine. The data is shown in FIG. 8B and suggests there is no drug-drug interaction between camonsertib and gemcitabine.

Patients were evaluated for treatment emergent adverse events (TEAE), and specifically for dose limiting toxicities (DLTs) within the first few weeks of treatment. Dose limiting toxicity was considered if it occurred during the first cycle and was deemed at least possibly related to study treatment. Given the high rate of DLTs in Arm 1 (40%, n=2) at the starting dose of camonsertib 80 mg QD (3 days on, 4 days off) and gemcitabine 1000 mg/m², additional cohorts were opened to evaluate lower starting doses of gemcitabine: 800, 600, and 400 mg/m². Despite the reduced starting dose of gemcitabine, the rate of DLTs did not significantly improve, with a cumulative DLT rate of 78% (n= 7/9) across these dose levels (Dose Level 1 through Dose Level-3). The most common related TEAE was neutropenia (n=6), which often occurred after the first dose of gemcitabine. For patients who remained on treatment, toxicities were managed with dosing holidays, schedule changes and/or dose reductions of either or both study drugs (FIG. 8C). In Arm 2, there was reduced frequency of DLTs 18% ( 4/22) and febrile/Grade 4 neutropenia. Table 4 below provides a summary of the most common treatment emergent adverse events.

TABLE 4
Most Common Treatment Emergent Adverse Events

| | Arm 1: cam 80 mg QD (3/4 or 2/5) + | | | Arm 2: cam 80 mg or 120 mg (2/5) + | | |
|---|---|---|---|---|---|---|
| | gem doses 400-1000 mg/m² | | | gem doses 100-200 mg/m² | | |
| Preferred | (N = 35) | | | (N = 27) | | |
| term, % | All grades | Grade 3 | Grade 4 | All grades | Grade 3 | Grade 4 |
| Neutropenia* | 66 | 34.3 | 25.7 | 52 | 25.9 | 11.1 |
| Fatigue | 46 | 0 | 0 | 63 | 7 | 0 |
| Anemia | 46 | 20 | 0 | 56 | 37 | 0 |
| Alopecia | 43 | 0 | 0 | 44 | 0 | 0 |
| Nausea | 46 | 0 | 0 | 37 | 6 | 0 |
| Thrombocytopenia | 37 | 9 | 0 | 41 | 6 | 0 |
| Pyrexia | 40 | 0 | 0 | 15 | 0 | 0 |
| Leukopenia | 29 | 20 | 0 | 26 | 11 | 0 |
| Vomiting | 22 | 0 | 0 | 30 | 0 | 0 |
| Chills | 26 | 0 | 0 | 15 | 0 | 0 |
| Stomatitis | 29 | 0 | 0 | 116 | 0 | 0 |

| | | |
|---|---|---|
| Dose Limiting | Neutropenia (N = 4), Anemia (N = 3), | Neutropenia (N = 3), Other (N = 2) |
| Toxicities | Other (N = 3) | |

*Neutropenia at preliminary RP2D: 50% (Grade 3+); 28.6% (Grade 4)

Of the 18 patients treated in these initial cohorts, four patients had clinical benefit (>14 wks on treatment). Three of these patients (Ovarian/ATM, Ovarian/BRCA1, Endometrial/PALB2) had confirmed RECIST partial responses. These patients started at camonsertin 80 mg and gemcitabine 1000, 800 and 400 mg/m² but due to toxicity, reduced to 200 mg/m² after which tolerability improved. Two of these patients (Ovarian/ATM and Ovarian/BRCA1) have been on study for more than one year, with partial responses maintained with a gemcitabine dose of 200 mg/m² for 29+ and 38+ weeks. The third patient (Endometrial/PALB2) whose starting dose camonsertib 80 mg and gemcitabine 400 mg/m² had a further response after reduction to 200 mg/m² on week 3, but withdrew at 18 weeks due to quality of life/tolerability concerns.

Given the continued toxicity at starting doses ≥400 mg/m² additional cohorts were opened which evaluated lower doses of gemcitabine (100 or 200 mg/m²) paired with 120 or 80 mg Camonsertib (2 days on/S days off). A new schedule was also evaluated, with gemcitabine administration on Days 1 and 15, and camonsertib (80 mg) on Days 1-2 and 15-16 on a 28-day cycle. This intermittent weekly schedule allows for hematological recovery in between gemcitabine doses and has been shown to have a more favorable toxicity profile in a subset of ovarian patients treated with gemcitabine and cisplatin (George B et al., Gynecologic Oncology, (104)3 (2007)).

Patients who started on treatment with gemcitabine doses of 100 or 200 mg/m² (21-day cycle) had a lower DLT rate (27%, n=15) and fewer Grade 4 related AEs, but the majority of patients ( 10/18) still required dose reductions by the start of the next cycle. In contrast, patients treated on the 1 week on/i week of schedule (28-day cycle, gemcitabine dose of 200 mg/m²) had no DL-Ts though there was also no evidence of efficacy ( 6/7 patients had progression (either clinical or RECIST) within 10 weeks of starting on study). Efforts are ongoing to refine the dose and dosing schedule to improve the tolerability of the combination, hence a result, two additional cohorts were recently opened, combining gemcitabine 400 mg/m² and camonsertib 80 mg (2 days on/days off or 3 days on/4 days off) on a 1 week on/i week off schedule.

The clinical benefit rate (RECIST or CA-125 [GCIG] response or treatment duration ≥16 weeks without progression) was found to be 48.7% ( 19/39) in evaluable patients, and 68.8% ( 11/16) in evaluable pts with gynecological cancers (FIG. 8D).

TABLE 5
Summary of Response Rates

| | | | | Enrolled | Final/ | Time on | | Best change |
|---|---|---|---|---|---|---|---|---|
| | Enrollment | Allelic | Prior Lines | dose | current dose | treatment | | in TL from |
| Tumor Type | gene | status | (N/Parp/gem) | (gem/cam) | (gem/cam) | (wks) | Response | baseline |
| Ovarian | ATM | subclonal | 3/Y/N | 1000 gem, | 200 gem, | 70+ | cPR | −52% |
| | | | | 80 cam | 80 cam | | | |
| | | | | (3/4) | (2/5) | | | |
| | | | | 2 wks on/ | 1on/1off | | | |
| | | | | 1 week off | | | | |
| | BRCA1 | monoallelic | 1/Y/N | 800 gem, | 200 gem, | 70+ | cPR | −31% |
| | | | | 80 cam | 50 cam | | | |
| | | | | (3/4) | (2/5) | | | |
| | | | | 2 wks on/ | 2 wks on/ | | | |
| | | | | 1 week off | 1 wk off | | | |
| | gBRCA1 | biallelic | 3/Y/N | 100 gem, | No change | 25 | CA-125 | +18% |
| | | | | 80 cam | | | | |
| | | | | (2/5) | | | | |
| | | | | 2 wks on/ | | | | |
| | | | | 1 wk off | | | | |
| | gBRCA1 | unknown | 5/Y/Y | 400 gem, | 300 gem, | 10+ | CA-125 | −9.1% |
| | | | | 80 cam | 80 cam | | | |
| | | | | (3/4) | (3/4) | | | |
| | | | | 1 wks on/ | 1 wks on/ | | | |
| | | | | 1 wk off | 1 wk off | | | |
| Endometrial | gPALB2 | unknown | 3/N/N | 400 gem, | 200 gem, | 17 | cPR | −64% |
| | | | | 80 cam | 80 cam | | | |
| | | | | (3/4) | (2/5), | | | |
| | | | | 2 wks on/ | 2 wks on/ | | | |
| | | | | 1 wk off | 1 wk off | | | |
| Breast | gBRCA1 | unknown | 3/Y/N | 200 gem, | 100 gem, | 22 | uPR^{a} | −30.5% |
| | | | | 80 cam | 80 cam | | | |
| | | | | (2/5) | (2/5) | | | |
| | | | | 2 wks on/ | 2 wks on/ | | | |
| | | | | 1 week off | 1 week off | | | |

^{a}PR unconfirmed due to progression of brain lesions though sustained reduction in TLs

The ctDNA molecular response in evaluable Arm 1 and 2 patients is shown in FIG. 8E. The on-treatment time-points were analyzed between 4-12 wks for best median response, and the median time point for data shown is 5 weeks. Similar rates of molecular response were found in both Arm 1 and Arm 2 patients.

One patient, a 59-year-old with high-grade serous ovarian cancer and ATM mutation (subclonal) had been treated with three lines of prior treatment (platinum and PARPi resistant) was enrolled at a dose of 80 mg camonsertib (3 day on and 4 days off) and 1000 mg/m² gemcitabine, on a 21-day schedule. Due to toxicity, four dose reductions occurred in first 21 weeks of the study. After a change to a 28-day schedule and observed progression, the dose was increased to final dose of 80 mg camonsertib (2 day on/5 days off) and 200 mg/m² gemcitabine, in a 28 day cycle. The duration of treatment was greater than 70 weeks. CA-125 response was observed at 11 weeks, with RECIST v1.1 confirmed PR at 12 weeks. Plots of the change in the target lesion size and CA-125 concentration are shown in FIGS. 8F-8G.

Camonsertib monotherapy has demonstrated clinical activity in patients with advanced solid tumors with ATRi-sensitizing alterations in DDR genes (Timothy et al., Proceedings of the American Association for Cancer Research Annual Meeting 2022; 2022 April 8-13). Camonsertib and gemcitabine are shown herein to synergize and that even sub-monotherapy dose levels of gemcitabine in combination with camonsertib at intermittent schedules demonstrate intriguing anti-tumor activity in tumors carrying ATRi-sensitizing alterations with the potential to increase the therapeutic window.

In the clinical trials, grade 3+ neutropenia was found to be the dominant drug-related toxicity. Introduction of the 1 wk on/1 wk off schedule enables neutrophil recovery in between gemcitabine doses. A preliminary RP2D is considered to be 80 mg camonsertib (D1-3, 15-17)+400 mg/m² gemcitabine or less (D1, 15), 28 d cycle.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of treating a cancer in a subject, the method comprising administering to the subject in need thereof a therapeutically effective amount of camonsertib and therapeutically effective amount of gemcitabine, wherein the therapeutically effective amount of gemcitabine is a subtherapeutic regimen of the gemcitabine.

2. The method of claim 1, wherein the gemcitabine is administered in a dosage ranging from 10 mg/m² to 400 mg/m².

3. The method of claim 2, wherein the dosage of gemcitabine is selected from a dosage of 10 mg/m², 25 mg/m², 50 mg/m², 75 mg/m², 100 mg/m², 125 mg/m², 150 mg/m², 175 mg/m², 200 mg/m², 250 mg/m², 275 mg/m², 300 mg/m², 325 mg/m², 350 mg/m², 375 mg/m², or 400 mg/m².

4. The method of claim 3, wherein the dosage of gemcitabine is 100 mg/m².

5. The method of claim 3, wherein the dosage of gemcitabine is 200 mg/m².

6. The method of claim 1, wherein the therapeutically effective amount of camonsertib is a subtherapeutic regimen of camonsertib.

7. The method of claim 1, wherein the camonsertib is administered in an amount ranging from 10 mg to 200 mg.

8. The method of claim 1, wherein the camonsertib is administered in an amount of 80 mg or 120 mg.

9. The method of claim 1, wherein the camonsertib and gemcitabine are administered to the subject in a treatment cycle comprising administration of the gemcitabine to the subject in an amount of 50 to 100 mg/kg once a week, and administration of the camonsertib to the subject in an amount of 5 to 30 mg/kg for three day 3 days followed by a period of 4 days without administration of camonsertib.

10. The method of claim 9, wherein the treatment cycle is repeated over a period of 28 days.

11. The method of claim 9, wherein the gemcitabine is administered in an amount of 50 mg/kg or 100 mg/kg.

12. The method of claim 9, wherein the camonsertib is administered in amount of 5 mg/kg, 10 mg/kg or 30 mg/kg.

13. The method of claim 1, wherein the camonsertib and gemcitabine are administered to the subject in a 28 day cycle comprising administration of the gemcitabine to the subject in an amount of 50 to 200 mg/m² on days 1 and 15, and administration of the camonsertib to the subject in an amount of 5 to 200 mg on days 1, 2, 15 and 16.

14. The method of claim 13, wherein the gemcitabine is administered in an amount of 100 mg/m² or 200 mg/m².

15. The method of claim 13, wherein the camonsertib is administered in amount of 80 mg or 120 mg.

16. The method of claim 1, wherein the camonsertib and gemcitabine are administered to the subject in a 28 day treatment cycle comprising administration of the gemcitabine to the subject in an amount of up to 400 mg/m² on days 1 and 14, and administration of the camonsertib to the subject in an amount of up to 80 mg on days 1, 2, 3, 15, 16 and 17.

17. The method of claim 1, wherein the cancer is a solid tumor with deleterious DNA damage response alterations.

18. The method of claim 1, wherein the cancer is associated with a loss of function of ATM, ATRIP, BRCA1, BRCA2, CDK12, CHTF8, FZR1, MRE11, NBN, PALB2, RAD17, RAD50, RAD51B/C/D, REV3L, RNASEH2A, RNASEH2B or SETD2.

19. The method of claim 1, wherein the cancer is associated with a loss of function of ATM, BRCA1, BRCA2, or PALB2.

20. The method of claim 1, wherein the cancer is a carcinoma, sarcoma, adenocarcinoma, leukemia, or melanoma.

21. The method of claim 1, wherein the cancer is selected from renal cell carcinoma, mature B-cell neoplasms, endometrial cancer, ovarian cancer, fallopian tube cancer, primary peritoneal cancer, colorectal cancer, skin cancer, small bowel cancer, non-small cell lung cancer, melanoma, bladder cancer, pancreatic cancer, head and neck cancer, mesothelioma, glioma, prostate cancer, breast cancer, or esophagogastric cancer.

22. The method of claim 21, wherein the cancer is ovarian, pancreatic, prostate, or lung cancer.

23. The method of claim 1, wherein the subject is a mammal.

24. The method of claim 1, wherein the subject is human.

25. The method of claim 1, wherein the administering results in a reduction of neutrophils of less than 15%.
